(19) United States
(12) Patent Application Publication
(10) Pub. No.: US 2026/0263717 A1
(43) Pub. Date: Sep. 10, 2026

(54) SYSTEMS AND METHODS FOR PROVIDING PERSONALIZED PRESSURE WAVEFORMS

(71) Applicant: ResMed Pty Ltd, Bella Vista, NSW (AU)

(72) Inventors: Andrew William GILLETT, Sydney (AU); Andrew CHAN, Sydney (AU); Michael James DENT, Sydney (AU)

(73) Assignee: ResMed Pty Ltd, Bella Vista, NSW (AU)

(21) Appl. No.: 19/166,198

(22) PCT Filed: Apr. 12, 2024
(86) PCT No.: PCT/AU2024/050346

§ 371 Date: Sep. 17, 2025

Related U.S. Application Data
(60) Provisional application No. 63/496,054, filed on Apr. 14, 2023.

(51) Int. Cl. A61M 16/00

(52) U.S. Cl. CPC A61M 16/024; A61M 2205/3303; A61M 2205/52

(57) ABSTRACT

The present technology includes systems, apparatus, and methods that provide personalization of pressure waveforms and can generate positive airway pressure (PAP), such as with a respiratory pressure therapy device (4000), in accordance with personalized pressure waveforms such as to improve comfort with therapy.

### 1 CROSS REFERENCE TO RELATED APPLICATIONS

This application claims the benefit of U.S. Provisional Patent Application Ser. No. 63/496,054, filed 14 Apr. 2023, the entire disclosure of which is hereby incorporated herein by reference.

### 1.1 FIELD OF THE TECHNOLOGY

The present technology generally relates to respiratory therapy systems and devices and adjustment of therapy parameters. More particularly, the technology concerns systems and methods for providing personalized pressure waveforms and generating positive airway pressure (PAP) in accordance with personalized pressure waveforms.

### 1.2 DESCRIPTION OF RELATED ART

Home-based respiratory therapy devices allow patients to receive respiratory treatments at the comfort of the patients' home. Some such devices can typically include automated algorithms that detect disordered breathing symptoms and in response, adjust treatment pressure in effort to alleviate the disordered breathing events and may even return to lower pressures after such events are alleviated. Such therapeutic changes to treatment pressure treat all patients alike and do well to address the detected respiratory/clinical events like flow limitation and obstructive apnea events but these changes to therapy pressure do not generally address the particular user's perceived comfort with the pressure therapy.

For many PAP users, the sensation of breathing with such therapy pressure can feel unnatural and uncomfortable, leading to low usage or even rejection of PAP therapy altogether. When a patient receives respiratory therapy with a pressure waveform that significantly differs from the patient's own unique breathing pattern and/or preferences, the patient may perceive the therapy received as uncomfortable. Respiratory therapy devices may permit some degree of adjustment to control parameters that concern delivery of therapy. However, such access to such changes are usually limited to authorized users such as clinicians. Moreover, existing respiratory pressure therapy devices may not typically generate any pressure therapy during such clinical setup as the clinician user adjusts the settings. As such, until use during a therapy session in a therapy mode, the patient does not feel the effect of a previously made adjustment, and given the separation, does not typically even appreciate what changes have been made. Indeed, such a process does not readily permit the patient to appreciate what was changed, such as from the comparative sensory perspective concerning multiple potential changes, or what might be a more desirable change for the patient.

Furthermore, even after any adjustments concerning therapy are made, the patient may still feel discomfort when receiving respiratory therapy from the respiratory therapy device. For example, individual patients may have a unique breathing pattern. Additionally, each patient may have different preferences with respect to aspects of delivered therapy that are perceived as comfortable or uncomfortable.

Existing respiratory therapy devices do not readily permit personalization to the therapy, such as with respect to the control parameters of the pressure waveform, in relation to the patient's unique breathing pattern and preferences. Thus, a need may exist for respiratory therapy devices and systems that provide a greater degree of personalization so as to increase patient comfort when receiving PAP therapy.

### 2 BRIEF SUMMARY OF THE TECHNOLOGY

The present technology is directed towards improved therapy systems and apparatuses that can provide for personalization of pressure waveforms and can generate positive airway pressure (PAP) in accordance with personalized pressure waveforms.

Some implementations of the present technology may include a system for providing a respiratory therapy to an airway of a user. The system may include one or more sensors configured to generate output signals conveying information related to one or more physiological parameters of a user, each of the one or more physiological parameters are indicative of a degree of user comfort with the respiratory therapy. The system may include a pressure generator configured to couple with a patient respiratory interface for delivery of the respiratory therapy to the airway of the user. The system may include a controller coupled to the pressure generator and comprising one or more processors. The controller may be configured to execute a waveform adjustment control loop. The controller may be configured to, during the waveform adjustment control loop: receive output signals from the one or more sensors during delivery of a pressurized flow of breathable gas to the airway of the user in accordance with a predetermined waveform, compare each of the one or more physiological parameters to a corresponding baseline value, adjust at least one waveform parameter of the predetermined waveform based on the comparison, wherein the adjustment is selected to reduce a difference between a value of at least one of the one or more physiological parameters and the corresponding baseline value for the at least one physiological parameter, and operate the pressure generator to generate a pressurized flow of breathable gas in accordance with the predetermined waveform as adjusted.

In some implementations, the controller may be configured to perform the waveform adjustment control loop iteratively for a predetermined period of time.

In some implementations, the at least one waveform parameter may comprise an inspiratory shape of the predetermined waveform. The controller may be configured to adjust the predetermined waveform such that the inspiratory shape of the waveform is linear. The controller may be configured to adjust the predetermined waveform such that the inspiratory shape is rounded. The at least one waveform parameter may comprise an expiratory shape of the predetermined waveform. The controller may be configured to adjust the predetermined waveform such that the expiratory shape of the waveform is linear. The controller may be configured to adjust the predetermined waveform such that the expiratory shape is rounded. The at least one waveform parameter may comprise a rise time of an inspiration phase of the predetermined waveform. The controller may be configured to adjust a duration of the rise time. The at least one waveform parameter may comprise a fall time of an expiration phase of the predetermined waveform. The controller may be configured to adjust a duration of the fall time. The at least one waveform parameter may comprise an inspiratory pressure trigger threshold of the predetermined waveform. The at least one waveform parameter may comprise an expiratory pressure trigger threshold of the predetermined waveform. The at least one waveform parameter may comprise a peak expiratory pressure of the predetermined waveform. The at least one waveform parameter may comprise a peak inspiratory pressure of the predetermined waveform.

In some implementations, the one or more physiological parameters may comprise one or more of flow rate, pressure, carbon dioxide, tidal volume, breathing rate, breathing effort, heart rate, and movement.

In some implementations, the one or more physiological parameters may comprise two or more of flow rate, pressure, carbon dioxide, tidal volume, breathing rate, breathing effort, heart rate, and movement.

In some implementations, the controller may be configured to store the predetermined waveform as adjusted in memory.

Some implementations of the present technology may include a system for providing a respiratory therapy to an airway of a user. The system may include a pressure generator configured to couple with a patient respiratory interface for delivery of the respiratory therapy to the airway of the user. The system may include a controller coupled to the pressure generator and comprising one or more processors. The controller may be configured to execute a waveform selection process. The controller may be configured to, during the waveform selection process: provide an indication to a user of delivery of a first waveform in association with operating the pressure generator to generate a pressurized flow of breathable gas in accordance with the first waveform for a first period of time, provide an indication to the user of delivery of a second waveform in association with operating the pressure generator to generate a pressurized flow of breathable gas in accordance with the second waveform for a second period of time, and with a user interface, and prompt for entry of an input selection between the indication of delivery of the first waveform and the indication of delivery of the second waveform.

In some implementations, the first waveform may be generated in accordance with a first set of one or more waveform parameters and the second waveform may be generated in accordance with a second set of one or more waveform parameters. At least one waveform parameter of the first set may be different from at least one waveform parameter of the second set. The different at least one waveform parameter may comprise any one of: an inspiratory shape, an expiratory shape, a rise time of an inspiration phase, a fall time of an expiration phase, an inspiratory pressure trigger threshold, an expiratory pressure trigger threshold, a peak inspiratory pressure, a peak expiratory pressure, an inspiratory pressure trigger threshold, and an expiratory pressure trigger threshold. The controller may be configured to generate a third waveform based on the entered input selection of the user. After generating the third waveform, during the waveform selection process, the controller may be configured to repeat the waveform selection process with (a) the waveform selected by the user between the first waveform and the second waveform and (b) the third waveform. The controller may be configured to perform the waveform selection control process iteratively for a predetermined period of time or for a predetermined number of control selection cycles. The controller may be configured to prompt for an indication from the user that the first waveform or the second waveform is at an acceptable level of comfort. The controller may be configured to discontinue the waveform selection control process in response to the indication. The controller may be configured to store the first waveform or the second waveform in memory based on the indication from the user.

Some implementations of the present technology may include a system for providing a respiratory therapy to an airway of a user. The system may include one or more sensors configured to generate output signals conveying information related to one or more physiological parameters of a user, each of the one or more physiological parameters are indicative of aspects of a user's breathing. The system may include a pressure generator configured to couple with a patient respiratory interface for delivery of the respiratory therapy to the airway of the user. The system may include a controller coupled to the pressure generator and comprising one or more processors. The controller may be configured to: monitor output signals of the one or more sensors for a period of time, approximate the user's breathing pattern based on the monitored output signals, generate a personalized pressure waveform based on the approximation of the user's breathing pattern, and operate the pressure generator to generate a pressurized flow of breathable gas in accordance with the generated personalized pressure waveform.

In some implementations, the one or more sensors may comprise one or more of a pressure sensor, a flow sensor, a heart rate sensor, a carbon dioxide sensor, an accelerometer, and one or more EEG sensors. The one or more physiological parameters may comprise one or more of pressure, flow rate, heart rate, carbon dioxide, chest body movement, and lung bioimpedance. The controller may be configured to store one or more parameters of the personalized pressure waveform in memory.

Of course, portions of the aspects or implementations may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 2.1 BRIEF DESCRIPTION OF DRAWINGS

The present technology is illustrated by way of example, and not by way of limitation, in the figures of the accompanying drawings, in which like reference numerals refer to similar elements including:

FIG. 1A shows an example therapy apparatus for providing a respiratory therapy (e.g., positive airway pressure (PAP) such as bi-level or variable level CPAP or pressure support) to an airway of a user with an example user interface of the present technology such as in a therapy-active user adjustment feedback mode while permitting the user to make manual adjustments to one or more therapy control parameters to the therapy being delivered;

FIG. 1B illustrates features of such a therapy apparatus with a wireless control device and one or more servers in some versions of the present technology;

FIG. 1C is an illustration of a target waveform and waveform parameters of the target waveform in accordance with the present technology;

FIG. 1D shows another example environment of a system for providing a therapy to an airway of a user, where the user may adjust therapy settings of the system through a user interface of a wireless device.

FIG. 2A is an illustration of transitions of an example graphical user interface, such as on a display screen or touch screen of the therapy apparatus or wireless control device of FIG. 1A or 1B, showing a target pressure waveform that can be visually manipulated or adjusted by the user to correspondingly achieve parameter adjustments, and its visual response to the user as the user manipulates or adjusts the target pressure waveform and thereby adjusting the corresponding or associated therapy control parameters, such as in a therapy-active user adjustment feedback mode of the present technology.

FIG. 2B is an illustration of a graphical user interface visually presenting running waveforms in an overlaying fashion, such as on a display screen of the therapy apparatus or wireless control device of FIG. 1A or 1B, such that the waveforms (e.g., pressure and flow rate) correspond with the pressure delivered by the respiratory apparatus and the flow rate of the patient as detected by the therapy apparatus, which may be presented in a therapy-active user adjustment feedback mode of the present technology.

FIG. 2C is another illustration of a graphical user interface, such as on a display screen of the therapy apparatus or wireless control device of FIG. 1A or 1B, visually presenting a transition in shape of the pressure waveform, which may be presented in a therapy-active user adjustment feedback mode of the present technology in response to a manual change to a control parameter made by a user, such as with a control (button or icon) of a user interface described herein.

FIG. 3A illustrates adjustment to waveform shape parameters in accordance with the present technology.

FIG. 3B illustrates adjustment to waveform time parameters, such as rise time and fall time, of a pressure waveform in accordance with the present technology.

FIG. 3C illustrates adjustment to waveform timing parameters of a pressure waveform in accordance with the present technology.

FIG. 3D illustrates adjustment to an expiratory pressure relief parameter (or multiple associated parameters) of a pressure waveform in accordance with the present technology.

FIG. 3E illustrates three unique breathing patterns in accordance with the present technology.

FIG. 4A is an illustration of a graphical user interface, such as on a display screen of the therapy apparatus or wireless control device of FIG. 1A or 1B, showing manually adjustable visual features or feature icons, on a target pressure waveform, that may be adapted for implementing therapy parameter adjustment, such as in a therapy-active user adjustment feedback mode of the present technology.

FIG. 4B is another illustration of a graphical user interface, such as on a display screen of the therapy apparatus or wireless control device of FIG. 1A or 1B, showing the user touching one visual feature or feature icon on the target pressure waveform of the display for selection of therapy parameter adjustment associated with the visual feature or icon, such as in a therapy-active user adjustment feedback mode of the present technology.

FIG. 4C is an illustration of a graphical user interface presenting adjustment icons or arrow icons, such as in response to the selection of FIG. 4B, that may be adapted for implementing parameter adjustments associated with a selected visual feature or feature icon, such as the one selected in FIG. 4B, such as in a therapy-active user adjustment feedback mode of the present technology.

FIG. 5 is a flow diagram of an example waveform selection control loop, such as to permit user selection between waveforms after experiencing two different waveforms, in accordance with the present technology.

FIG. 6A is an illustration of an example waveform adjustment control loop in accordance with the present technology.

FIG. 6B is a flow diagram of the example waveform adjustment control loop of FIG. 6A.

FIGS. 7A-7C illustrate an example of a breathing pattern approximation and personalized waveform generation process in accordance with the present technology.

FIG. 7D illustrates a flow diagram of the breathing pattern approximation and personalized waveform generation process of FIGS. 7A-7C.

FIG. 8A shows an example system in accordance with the present technology. A patient 1000 wearing a patient interface 3000 receives a supply of pressurized air from an RPT device 4000. Air from the RPT device 4000 is humidified in a humidifier 5000, and passes along an air circuit 4170 to the patient 1000. A bed partner 1100 is also shown.

FIG. 8B shows an RPT device 4000 in use on a patient 1000 with a nasal mask 3000.

FIG. 8C shows an RPT device 4000 in use on a patient 1000 with a full-face mask 3000.

FIG. 9 shows an example non-invasive patient interface 3000 in the form of a nasal mask.

FIG. 10A shows an RPT device 4000 in accordance with one form of the present technology.

FIG. 10B shows a schematic diagram of the pneumatic circuit of an RPT device 4000 in accordance with one form of the present technology. The directions of upstream and downstream are indicated.

FIG. 10C shows a schematic diagram of the electrical components of an RPT device 4000 in accordance with one aspect of the present technology.

FIG. 10D shows a schematic diagram of the algorithms 4300 implemented in an RPT device 4000 in accordance with an aspect of the present technology. In FIG. 10D, arrows with solid lines indicate an actual flow of information, for example via an electronic signal.

FIG. 10E is a flow chart illustrating a method 4500 carried out by the therapy engine module 4320 of FIG. 10D in accordance with one aspect of the present technology.

FIG. 11 shows a humidifier 5000.

### 3 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 1. Pressure Support System

The present technology relates to a system or apparatus for providing therapy such as a pressure or flow therapy to an airway of a user. The system is configured to personalize (or facilitate personalization of) a pressure waveform to an individual user, which typically targets changes that provide greater user comfort for a particular user, as opposed to targeting changes that provide therapeutic improvements. In this way, a pressurized flow provided by the system to the user in accordance with the personalized pressure waveform can provide a level of personal customization that can increase patient comfort, which can lead a patient to greater therapy compliance. As described in greater detail below, the system may be configured to personalize a pressure waveform to the user in several ways including by manual adjustment and/or in an automated manner according to the present technology. As discussed in more detail herein, such systems may, for example, be configured according to any of the following:

Manual Self-Optimisation: A user interacting directly through a user interface (e.g., on a local therapy device such as also with an external control application) by changing setting(s) and experiencing them until their most comfortable combination of settings is identified.

Manual Optimisation with Trained Person: A user interacting with a trained person, such as with a user interface that enables the trained person to operate the device to allow the user to experience a series of different A or B tests (e.g., consider waveform A or waveform B) with the user experiencing and selecting their preference for A or B. Additionally, the trained person can listen to comments made by the user after each test. Based on the user preference and comments one or more settings can be changed with any A-B Test. The A-B tests may continue until the user's most comfortable combination of settings are identified and entered/selected.

Guided Self-Optimisation: User interacts directly through an automated user interface (a local therapy device such as also with an external control application), a series of such A-B tests (e.g., consider waveform A or waveform B) with the user experiencing and selecting their preference for A or B with the user interface. One or more settings are changed with each of the A-B tests. The A-B tests can continue until the user's most comfortable combination of settings are identified and stored (e.g., in memory). Such A-B tests may be staged at different times such as before or after different sessions such that they may be completed or performed before an initial use of the therapy device and/or any subsequent uses.

Automated Optimisation: A therapy device includes an automated comfort determination control loop where no direct user input is required on a user interface. The system may continually or periodically make changes based on comfort related inputs from one or more sensors (e.g., flow, pressure, breathing effort, heart rate etc.) to determine which settings changes improve comfort. Such automated optimization may run repeatedly or continuously.

Implementations of such systems may be considered in relation to the following passages concerning a pressure therapy device.

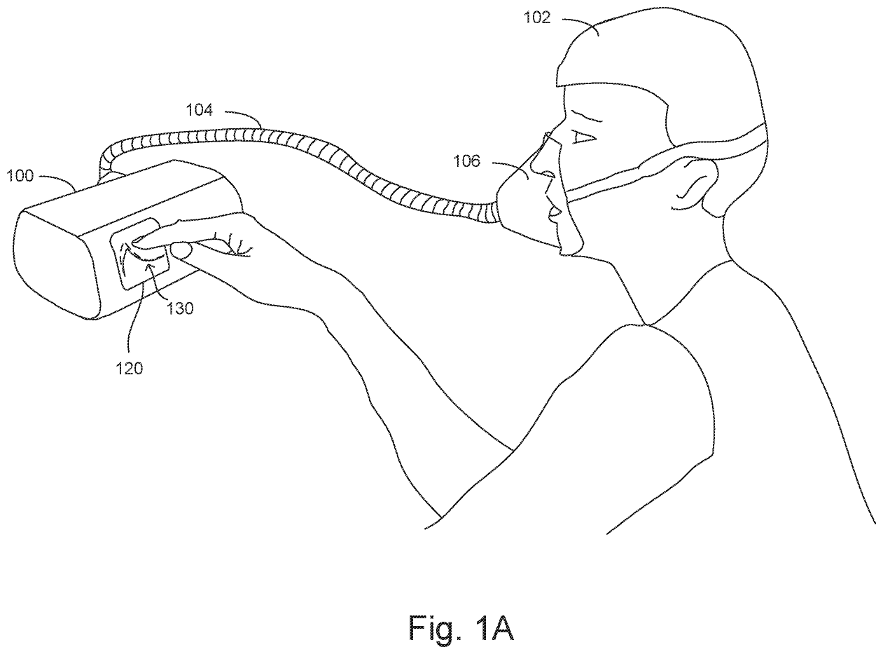

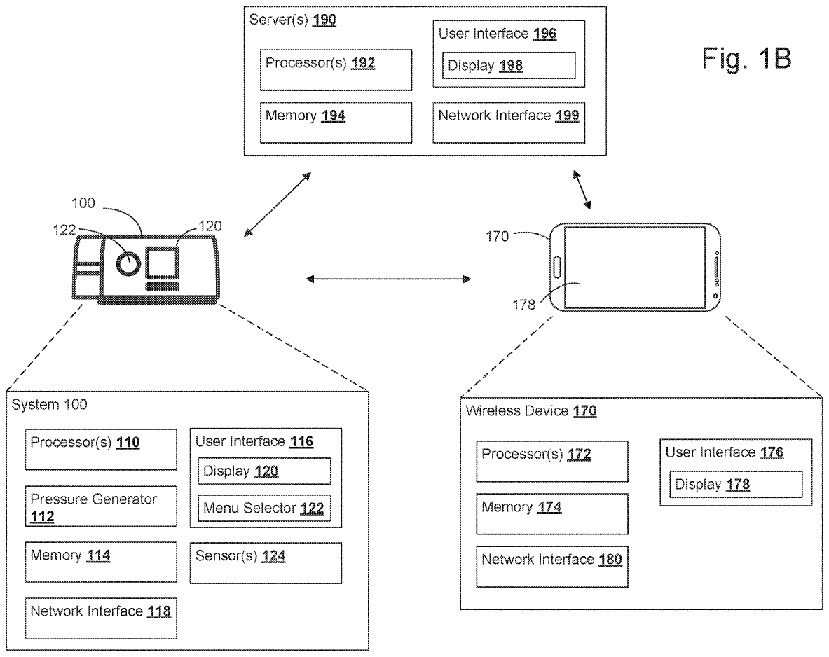

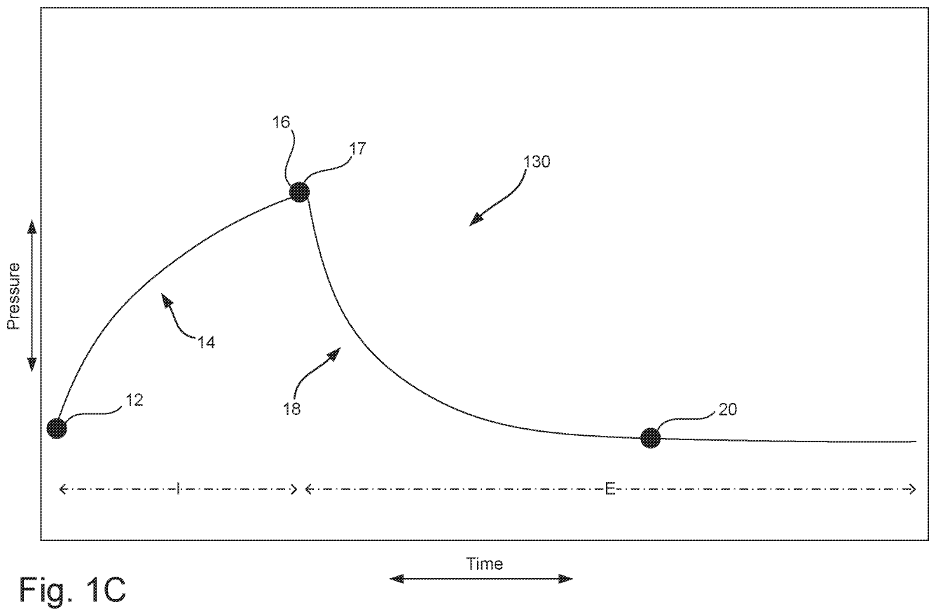

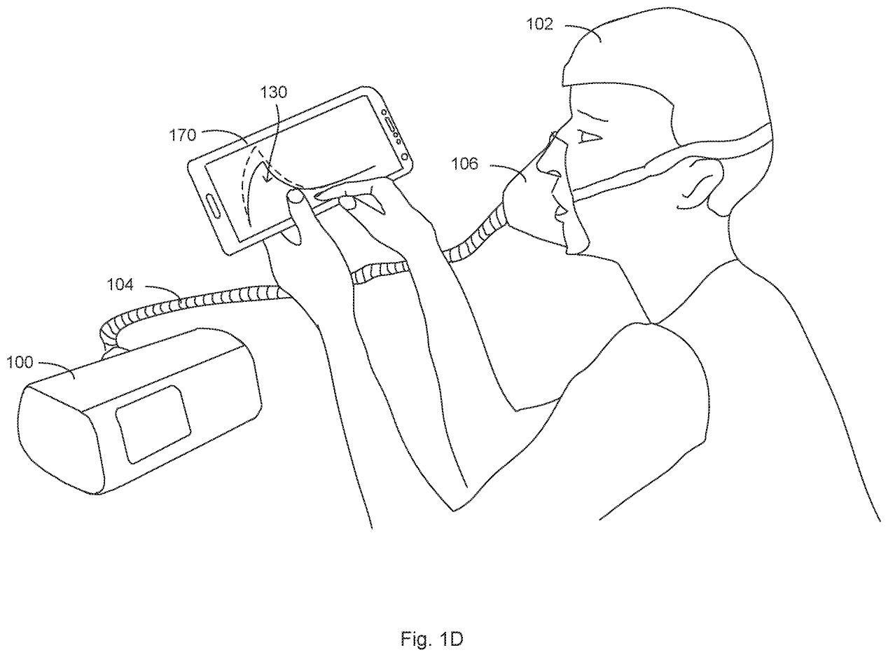

FIG. 1A shows an example environment of a system 100 that may be configured to provide pressure therapy, such as pressure support, to an airway of a user 102. In some implementations, the system 100 may include a respiratory therapy device that provides respiratory treatment to the user 102. For example, the system 100 may include a respiratory pressure therapy (RPT) device, such as any of the RPT devices describe in more detail herein. The system 100 may provide a flow of breathable gas to the user at a controlled pressure(s) and/or controlled flow rate(s). A patient interface 106, such as a respiratory interface or mask, and an air flow conduit 104 may be used to interface the system 100 to the user 102. Depending on the therapy to be applied, the patient interface 106 may form a seal, e.g., with a face region of the user 102, to facilitate the delivery of gas at a pressure at sufficient variance with ambient pressure to effect therapy.

As shown in FIG. 1B, the system 100 may have one or more of the following: a controller, e.g., comprising one or more processor(s) 110, operatively coupled to a pressure generator 112, one or more memory 114, a user interface 116, a network interface 118 and one or more sensors 124, among others.

The user interface 116 may include one or more of the following: a display 120 for presenting a graphical user interface and one or more selectors 122, such as menu selectors, which may be physical (e.g., knob, button, switch, etc.) or virtual (e.g., icon) components. The user interface 116 may be controlled by and exchange information with processor(s) 110. The graphical user interface may be generated by processor(s) 110 (e.g., based on instructions in memory 114) and displayed to the user via display 120. Processor(s) 110 may receive user inputs provided to interface 116. The selector(s) 122 may, for example, take the form of a knob or a button, which may be manipulated by the user 102 to operate the system 100. For example, the user 102 may manipulate the selector(s) 122 to navigate and/or select menus displayed in the display 120 or to make selections responsive to prompts provided to the user by processor(s) 110 and displayed on display 120. Optionally, such selectors 122 may be moved by touch to change values associated with the parameters, which may be visualized by changes to a waveform on the display. Thus, the display 120 may have a touch screen. The network interface 118 may have one or more transceivers, such as a Bluetooth transceiver, a cellular transceiver and a Wi-Fi communication transceiver. The network interface 118 may also be configured to wired communication with other systems or devices.

The sensor(s) 124, such as any of the sensors described in more detail herein, may be configured to sense and generate output signals conveying information related to therapy and/or physiological parameters of the user 102, such as, aspects of breathing of the user 102 or other physiological parameters, such as physiological parameters that are indicative of the user's comfort with the use of the therapy device. Information related to the user's breathing may include, but not limited to, a flow rate of the pressurized flow of breathable gas and/or a pressure of breathable gas at the user's mouth. Other physiological parameters that may be sensed by the sensor(s) 124 may include carbon dioxide (CO₂) concentrations, e.g., in the patient interface 106 and/or conduit 104, heart rate, breathing rate, movement (e.g., chest movement), bioimpedance of the lungs, etc. The processor(s) 110 may determine other physiological parameters such as tidal volume and transpulmonary pressure of the user based on the information in the output signals.

The pressure generator 112 may be configured to generate, under the control of a controller comprising one or more processor(s) 110, such as a controller described in more detail herein, a pressurized flow of breathable gas for delivery to the airway of the user 102 according to a target pressure waveform. The pressurized flow of breathable gas generated by pressure generator 112 may be provided from an outlet of pressure generator 112, through air flow conduit 104, to patient interface 106, and thereby to the airway of the user 102. The target pressure waveform, such as pressure values and/or one or more parameters and/or functions for producing such waveforms, may be stored in memory 114. FIG. 1C illustrates an example target pressure waveform 130 based on which the pressurized flow of breathable gas is generated. The target pressure waveform 130 may represent varying pressure of the flow of breathable gas that the pressure generator 112 aims to produce. The target pressure waveform 130 may include an inspiratory pressure or inspiratory positive airway pressure (IPAP), shown by “I” in FIG. 1C, that may assist or be associated with the user's inspiration. The target pressure waveform 130 may also include an expiratory pressure or expiratory positive airway pressure (EPAP), shown by “E”, that may assist or be associated with the user's expiration. Such pressures may or may not be therapeutic but the systems described herein are configured for targeting pressures (e.g., pressure versus time profiles) that are chosen especially for a particular user's comfort.

It is to be appreciated that system 100 may store additional target pressure waveforms in memory 114. For example, system 100 may store, in addition to target pressure waveform 130, a bi-level waveform (square waveform), that may be used. Memory 114 may store a plurality of different pressure waveforms as will be described in greater detail below.

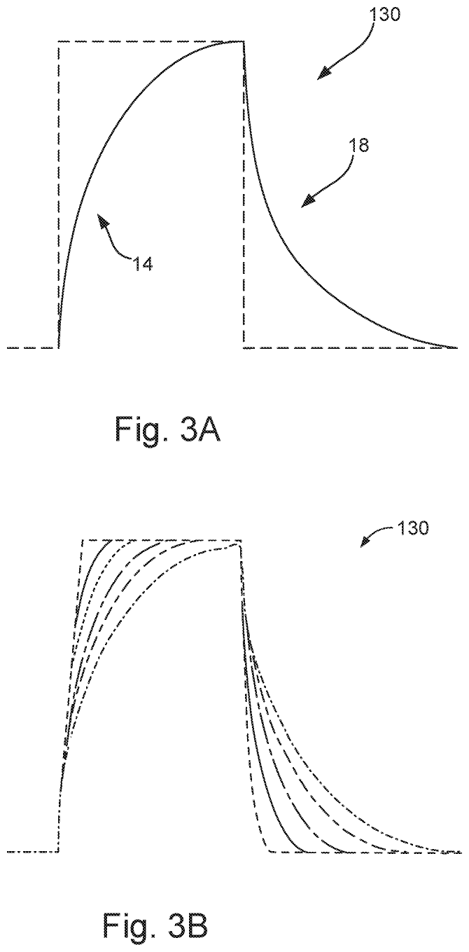

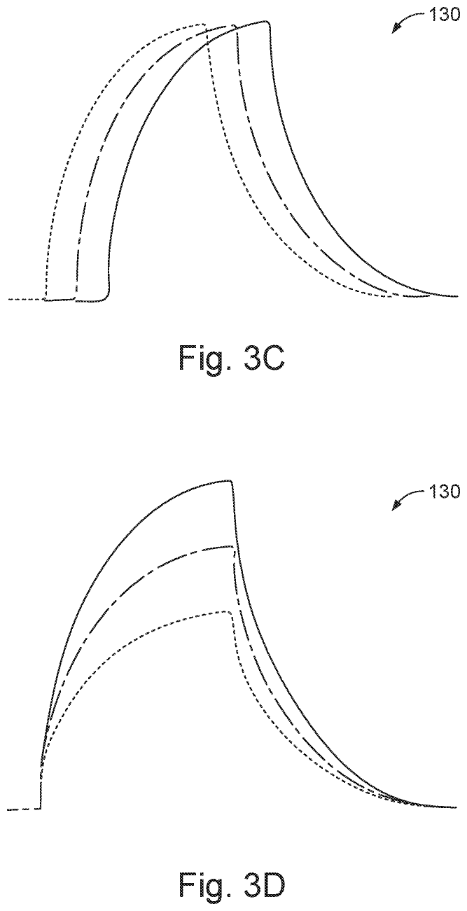

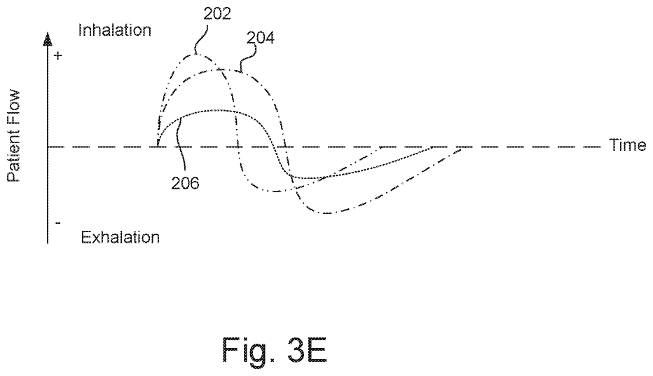

It will be understood that different user(s) 102 may have their own natural breathing pattern, i.e., the breathing pattern of each user that may be unique with respect to one or more breathing aspects. For example, referring to FIG. 3E, breathing patterns 202, 204, 206 that are unique to three different users are represented as air flow (flow rate) through each user's airway during the inhalation and exhalation phases of breathing. Considering the uniqueness of each person's natural breathing pattern which is most likely a comfortable waveform for the person, a single pressure waveform 130 delivered from a respiratory therapy system, such as system 100, may not be comfortable for each individual's breathing. In other words, the shape of the pressure waveform 130 may not match the unique shape and other aspects of each user's airflow. Additionally, the pressure waveform 130 may not match the user's subjective comfort preferences.

Systems, such as system 100, may also include some comfort related parameters for adjusting such waveforms such as a ramp feature and Expiratory Pressure Relief (EPR), which aim to help users acclimatize to PAP by reducing exhalation effort. For example, when a ramp feature is activated, processor(s) 110 may control a pressure generator 112 to start delivering a pressurized flow of breathable gas at a lower pressure (such as in relation to a peak inspiratory pressure) than a prescribed therapy pressure (e.g., a prescribed peak inspiratory pressure or an IPAP) at the beginning of a therapy session, with the delivered pressure gradually increased to the prescribed pressure later in the session. In this way, the user may gradually adjust to the sensation of pressure therapy. When the EPR feature is activated, processor(s) 110 may control the pressure generator 112 to reduce the pressure on exhalation (e.g., an EPAP) by up to 3cmH₂O, which can increase the subjective comfort of the user. The EPR feature may be used concurrently with the ramp feature or separately after the ramp feature is deactivated.

However, despite the use of such comfort related features such as the EPR feature and the ramp feature, individual user's 102 may still find PAP therapy to feel uncomfortable. For example, the shape (e.g., profile) or other waveform parameters of the target waveform 130 may not sufficiently match or compliment the user's natural breathing pattern. Moreover, the 3cmH₂O max pressure relief of the EPR feature may not provide enough relief for some users.

The present technology provides a system 100 with several different features and processes for providing a personalized waveform to the user that increases user comfort based on the user's unique breathing pattern and subjective preferences with respect to the pressure waveform used.

### 1.1 Adjustable Waveform Parameters

Thus, a controller may personalize or facilitate personalization of a target waveform for user comfort and permit selection of such a personalized target waveform. For example, a target pressure waveform, such as waveform 130, may be characterized by a plurality of waveform parameters that may be adjusted for the user's particular comfort. For example, to personalize a pressure waveform to the user, processor(s) 110 of system 100 may be configured to selectively adjust these waveform parameters to generate a personalized pressure waveform that is more comfortable for the user based on the user's unique or natural breathing pattern and/or subjective preferences. As will be described in greater detail below, system 100 may be configured to allow for manual, professionally guided, automatically self-guided, and/or automated personalization of the target pressure waveform.

The waveform parameters that are adjustable by processor(s) 110 may be categorized into four groups: waveform shape parameters, waveform time parameters, waveform timing parameters, and pressure relief parameters. It is to be appreciated that there may be overlap between the parameters in each of these groups and one or more parameters may be categorized in multiple groups. The groupings are not meant to be limiting. The waveform parameters may include, but are not limited to, any one or more of an inspiratory pressure trigger threshold, an inspiratory pressure shape, a peak inspiratory pressure peak, an expiratory pressure trigger threshold, an expiratory pressure shape, a peak expiratory pressure, inspiratory rise time, and expiratory fall time. Each of these waveform parameters may further comprise or be characterized by one or more (waveform) parameters that determine their adjustment. Moreover, there may be overlap between some of the waveform parameters. Several waveform parameters are discussed in detail below.

### 1.1.1 Waveform Shape Parameters

Waveforms shape parameters comprise parameters that, when adjusted, alter the shape of the inspiratory or expiratory portion of the target waveform 130.

Inspiratory pressure shape 14 (shown in FIG. 1C) may refer to a waveform parameter that determines the shape of the inspiratory pressure curve from the start of the inspiratory pressure to the peak inspiratory pressure. Processor(s) 110 may control the pressure generator 112 to increase the pressurized flow of breathable gas to the peak inspiratory pressure according to the inspiratory pressure shape 14. The inspiratory pressure shape 14 may correlate to the rise time of the inspiratory pressure from the beginning (e.g., an end expiratory pressure) to the peak inspiratory pressure. The inspiratory pressure shape 14 may control how fast or slow the inspiratory pressure rises to the peak inspiratory pressure.

The inspiratory pressure shape 14 may exhibit one or more of the following patterns: a linear line, a smooth curve (e.g., based on an exponential function that is adjustable by processor(s) 110), or a square-like curve. The inspiratory pressure shape may include a parameter that determines a slope or smoothness of the inspiratory pressure shape. In one example, by adjusting the inspiratory pressure shape parameter 14, such as by adjustment of a selector of the user interface 116 or automatic adjustment by processor(s) 110, the inspiratory pressure shape 14 may transform from one form to another, such as from a smooth curve to a square-like curve. In some aspects, processor(s) 110 may be configured to adjust the degree of linearity of the inspiratory pressure shape 14. For example, processor(s) 110 may adjust the shape to be more linear (i.e., to take a straighter/shorter path between the beginning of inspiration and the peak inspiratory pressure) or to be less linear and more curved (i.e., to increase the convexness of the inspiratory pressure shape 14).

Expiratory pressure shape 18 (shown in FIG. 1C) may refer to the shape of the expiratory pressure curve from the end of the inspiratory pressure to the peak expiratory pressure. Processor(s) 110 may control the pressure generator 112 to decrease the pressurized flow of breathable gas to the peak expiratory pressure according to the expiratory pressure shape 18. The expiratory pressure shape 18 may correlate to the fall time of the expiratory pressure from the end of the inspiratory pressure to the peak expiratory pressure. The expiratory pressure shape 18 may control how fast or slow the expiratory pressure drops in expiration.

The expiratory pressure shape 18 may exhibit one or more of the following patterns: a linear line, a smooth curve (e.g., based on an exponential function that is adjustable by processor(s) 110), or a square-like curve. The expiratory pressure shape 18 may include a parameter that determines a slope or smoothness of the expiratory pressure shape. In one example, by adjusting this parameter, the expiratory pressure shape 18 may transform from one form to another, such as from a smooth curve to a square-like curve, or vice versa. In some aspects, processor(s) 110 may be configured to adjust the degree of linearity of the expiratory pressure shape 18. For example, processor(s) 110 may adjust the shape to be more linear (i.e., to take a straighter/shorter path between the end of inspiratory pressure and the peak expiratory pressure) or to be less linear and more curved (i.e., to increase the concaveness of the expiratory pressure shape 18).

Referring to FIG. 3A, adjustment of the both the inspiratory pressure shape 14 and the expiratory pressure shape 18 is shown from a square curve to a smooth “saw-tooth” curve. It is to be appreciated that processor(s) 110 may be configured to adjust shapes 14, 18 independently. In this regard, a user may prefer a smooth shape on inspiration but a linear shape on expiration, or vice versa. Alternatively or additionally, processor(s) 110 may be configured to adjust shapes 14, 18 together, i.e., to perform the same type of adjustment to each of shapes 14, 18 (e.g., to make each of 14, 18 more rounded or more linear in shape).

Additionally, it is to be appreciated that adjustment to the shapes 14, 18 may also cause adjustment to other waveform parameters, such as rise time and fall time.

### 1.1.2 Waveform Time Parameters

Waveform time parameters comprise parameters that, when adjusted, alter the time it takes for pressure to increase on inspiration and the time it takes for pressure to decrease on expiration. The waveform time parameters may comprise the inspiratory rise time of the inspiratory pressure from the beginning (e.g., an end expiratory pressure) to the peak inspiratory pressure and the expiratory fall time of the expiratory pressure from the end of the inspiratory pressure to the peak expiratory pressure. An individual user may prefer a fast rise time on inspiration and a slower fall time on expiration, or vice versa. The waveform time parameters also include waveform parameters relating to rise and fall time and/or affecting rise and fall time.

The inspiratory rise time may be adjusted by processor(s) 110 by adjustment of one or more waveform parameters. For example, as described above, inspiratory pressure shape 14 may control how fast or slow the inspiratory pressure rises to the peak inspiratory pressure. Moreover, a “peak time” parameter of the peak inspiratory pressure 16 (shown in FIG. 1C) may affect the inspiratory rise time. The peak inspiratory pressure 16 and the peak time are described in greater detail below. Thus, any of these parameters may be adjusted by processor(s) 110 to adjust the rise time.

The expiratory fall time may be adjusted by processor(s) 110 by adjustment of one or more waveform parameters. For example, as described above, the expiratory pressure shape 18 may control how fast or slow the expiratory pressure drops in expiration. Moreover, a “peak” time parameter of the peak expiratory pressure 20 (shown in FIG. 1C) may affect the expiratory fall time. The peak expiratory pressure 20 and the peak time will be described in greater detail below. Thus, any of these parameters may be adjusted by processor(s) 110 to adjust the fall time.

The peak inspiratory pressure 16 may include a first parameter that controls when to generate the peak inspiratory pressure 16, which may be referred to as the time of the peak inspiratory pressure, or simply referred to as the peak time. The peak time may represent when the peak supply is stopped. For example, the peak time may indicate when to stop inspiratory pressure delivery function and when to change from an inspiratory pressure delivery function (e.g., a pressure rise function) to an expiratory pressure delivery function (e.g., a pressure decline function). The peak time may also indicate when the peak supply is achieved within a particular point of time within the patient's detected respiratory cycle, such as in relation to a determined phase of a patient's respiratory cycle as described in more detail herein. The peak time may affect the inspiratory rise time. For example, increasing the peak time may slow down the inspiratory pressure rise time, whereas decreasing the peak time may reduce the inspiratory pressure rise time. Based on the peak time, processor(s) 110 may control the pressure generator 112 to adjust the function/equation of inspiratory pressure delivery so that the pressure rises to the peak point of the inspiratory cycle by the desired time.

The peak inspiratory pressure 16 may include a second parameter that controls an amplitude of the peak inspiratory pressure, which may represent the amount of pressure supplied by the pressure generator 112 at the peak time (e.g., an IPAP pressure). This parameter may also concern the pressure rise function as the inspiratory pressure delivery function approaches the peak inspiratory pressure 16.

The peak expiratory pressure 20 may include a first parameter representing the amplitude of the minimum expiratory pressure generated by the pressure generator 112 during the user's expiration, or how far the pressure falls during the user's expiration. This parameter may concern the pressure decline function as the expiratory pressure delivery function approaches the peak expiratory pressure 20, which may be an ambient pressure or other reduction in pressure from the peak inspiration pressure 16.

The peak expiratory pressure 20 may include a second parameter indicating when to generate the peak expiratory pressure 20, which may be referred to as the time of the peak expiratory pressure. This peak time may indicate when to stop expiratory pressure delivery function. This peak time may also indicate when the peak expiratory pressure 20 is achieved within the detected respiratory cycles such as in relation to a determined phase as described in more detail herein. Based on this peak time, the pressure generator 112 may adjust the expiratory pressure delivery function so that the pressure falls to the peak point in the expiratory cycle by the desired time. This peak time may affect the expiratory fall time. For example, increasing the peak time may slow down the expiratory pressure fall time, whereas decreasing the peak time may reduce the expiratory pressure fall time.

Referring to FIG. 3B, adjustment to the inspiratory rise time and expiratory fall time (by adjusting one or more of the parameters described above) is shown.

### 1.1.3 Waveform Timing Parameters

Waveform timing parameters comprise parameters that define when pressure starts to increase on inspiration and when pressure starts to decrease on expiration. For example, an individual user may prefer the pressure to start increasing pressure earlier and decrease or drop off later, or vice versa. In one aspect, the waveform timing parameters comprise an inspiratory pressure trigger threshold (IPTT) 12 and an expiratory pressure trigger threshold (EPTT) 17.

The IPTT 12 may be a parameter that indicates when the pressure generator 112 starts to generate an inspiratory pressure to assist the user's inspiration during the user's inspiration cycle. The IPTT 12 may indicate how quickly the pressure generator 112 changes from generating the expiratory pressure to generating the inspiratory pressure in relation to the user's breathing cycle. In one aspect, the pressure generator 112 may not start generating the inspiratory pressure at the beginning of the user's inspiratory cycle. Rather, the pressure generator 112 may delay the inspiratory pressure generation until a later point in time of the user's inspiratory cycle or start at a time shortly preceding the start of the patient's inspiration.

The IPTT 12 may be a flow value, defined relative to the user's inspiratory flow as detected by the sensor(s) 124. In one example, the IPTT 12 may be set to a value such as 4.5 L/min. In this example, once the user's inspiratory flow reaches the IPTT 12 value, the pressure generator 112 may start to generate the inspiratory pressure. The IPTT 12 may serve to delay or expediate the start of the inspiratory pressure generation. For instance, increasing the value of IPTT 12 may delay the start of the inspiratory pressure generation, whereas decreasing the value of the IPTT 12 may expediate the start of the inspiratory pressure generation.

Alternatively, the IPTT 12 may be a pressure value, defined relative to the user's inspiratory pressure at the mask 106 as detected by the sensor(s) 124. In one example, the IPTT 12 may be a threshold value specifying a predetermined pressure in the mask 106 such as falling pressure indicative of patient inspiration. When the sensor(s) 124 detects the predetermined pressure in the mask 106, the pressure generator 112 may be triggered to start generating the inspiratory pressure. In this example, increasing the value of IPTT 12, which entails increasing the predetermined pressure, may delay the start of the inspiratory pressure generation, whereas decreasing the value of the IPTT 12, which entails decreasing the predetermined amount of pressure drop, may expediate the start of the inspiratory pressure generation.

The EPTT 17, or expiratory pressure cycle threshold, may be a parameter that controls when the pressure generator 112 starts to control a pressure reduction to assist the user's expiration during the user's expiration cycle. The EPTT 17 may indicate how quickly the pressure generator 112 changes from generating the inspiratory pressure to generating the expiratory pressure in relation to the user's breathing cycle.

The EPTT 17 may be a flow value, defined relative to the user's expiratory flow as detected by the sensor(s) 124, which may be in relation to a phase determination as described in more detail herein. In one example, once the user's expiratory flow reaches the EPTT 17 value, the pressure generator 112 may start to control a pressure reduction. The EPTT 17 may serve to delay or expediate the start of pressure reduction (e.g., pressure decline function). For instance, increasing the value of EPTT 17 may delay the start of pressure reduction, whereas decreasing the value of the EPTT 17 may expediate the start of pressure reduction.

Alternatively, the EPTT 17 may be a pressure value, defined relative to the user's expiratory pressure as detected by the sensor(s) 124 at the mask. In one example, the EPTT 17 may be a threshold value specifying a predetermined pressure increase in the mask. When the sensor(s) 124 detects the predetermined pressure increase in the mask, the pressure generator 112 may be cycled to start pressure reduction. In this example, increasing the value of EPTT 17, which entails increasing the predetermined amount of pressure increase, may delay the start of pressure reduction, whereas decreasing the value of the EPTT 17, which entails decreasing the predetermined amount of pressure increase, may expediate the start of pressure reduction.

Referring to FIG. 3C, adjustment to the IPTT 12 and EPTT 17 is shown.

### 1.1.4 Expiratory Pressure Relief (Epr)

The waveform parameters may further include expiratory pressure relief (EPR) adjustment. As descried above, EPR adjustment may be limited in existing respiratory therapy systems to 3 cmH₂O. The system 100 of the present technology may permit this parameter to be adjusted beyond this limit to higher values (e.g., up to 6 cmH₂O or higher). In one aspect, the lowest pressure on exhalation may remain at 4 cmH₂O. Referring to FIG. 3D, adjustment to EPR parameter is shown. The EPR may be set to a parameter that is most comfortable to the user. For example, as shown, the EPR parameter or settings may be set to different settings (e.g., illustrated settings 1, 2, and/or 3) to adjust the level or degree of relief.

### 1.2 Pressure Waveform Personalization

Processor(s) 110 are configured to adjust any one or more of the pressure waveform parameters described above to personalize the pressure waveform to the individual user such that user comfort is increased. System 100 is configured to personalize the pressure waveform in several different ways.

### 1.2.1 Manual Optimization

In one aspect, system 100 is configured to permit manual adjustment by the user to the waveform parameters of the target pressure waveform 130, such as in relation to its parameters/functions, stored in memory 114. User interface 116 is configured to receive user input from the user to adjust any one or more of the waveform parameters of the target pressure waveform 130 described above. The user interacts directly through the user interface 116 changing each waveform parameter until their most comfortable combination of waveform parameter settings (e.g., values corresponding to each setting) is identified. As will be described in greater detail below, the manual adjustment may occur during a set-up configuration mode of system 100 where the system 100 generates a sensory response perceived by the user in real time or near real time (e.g., breath by breath adjustment such that any change made becomes effective upon the next user breath) as the user adjusts one or more of the waveform parameters. In this way, the user may make as many adjustments as necessary to the waveform parameters until the pressure waveform 130 becomes personalized and comfortable to the user. When the user is satisfied with the adjustments and the resulting waveform, the adjusted pressure waveform, including the waveform parameter values corresponding thereto, may be saved, e.g., in memory 114. The adjusted pressure waveform may be used at a later time (e.g., an operational mode of system 100, as described below) to provide a pressurized flow of breathable gas to the user in accordance with the adjusted, personalized pressure waveform stored in memory 114.

In one aspect, the user interface 116 includes one or more menus that the user can navigate via selector 122 to select the settings to adjust the waveform parameters of the pressure waveform 130. In association with the one or more menus and settings, the user interface 116 may present the user with descriptions in relation to each waveform parameter informing the user which parameters the user may attempt to adjust first and what the user may expect if a particular parameter is adjusted. The table below includes example information that may be included in the one or more menus and settings or waveform parameters that may be adjusted within user interface 116 by the user.

TABLE 1

| Personalization | Setting/ | |
|---|---|---|
| Category | Parameter | User Terminology |
| Waveform | Square | Liked jolt sensation on exhalation |
| Shape | (Bi-Level) | Sudden rush of air not natural |
| | | Would be better to have a gradual |
| | | drop in pressure |
| | Rounded | Gentle/Gentleness |
| | (Easy- | Smooth/Smoothness |
| | Breathe) | Prefer softer changes between inhale |
| | | and exhale |
| Waveform | Rise Time | Steep/Steep, Quick/Quicker . . . |
| Time | | on inhalation |
| | | Responds too fast on inhalation |
| | Fall Time | Steep/Steep, Quick/Quicker . . . |
| | | on exhalation |
| | | Pressure dropped off too quickly |
| | | Liked jolt sensation on exhalation |
| | | shorter exhales than what I want |
| Waveform | Trigger (when | Responds too fast on Inhalation |
| Timing | pressure starts | No bang of flow at beginning of |
| | rise on | inhalation when inhale, air doesn't |
| | inhalation) | not start till after |
| | Cycle (when | Exhalation feels laggy |
| | pressure starts | Like the transition between inhale and |
| | to fall on | exhale pause between exhale and inhale, |
| | exhalation) | inhale stops prematurely |
| Pressure | Expiratory | Sudden pressure drop |
| Relief | Pressure | Very high resistance on exhale |
| | Relief | Very soft exhalation, like breathing |
| | (EPR) | against nothing, really like it, |
| | EPR | At start of inhale the system is working |
| | Gentleness | with me, it is working with me, |
| | | easier to exhale |
| | | Higher exhalation resistance |

### 1.2.1.1 Visual Adjustment

The system 100 may additionally or alternatively be configured to provide enhanced visual adjustment features for manually adjusting the waveform parameters and personalizing the pressure waveform.

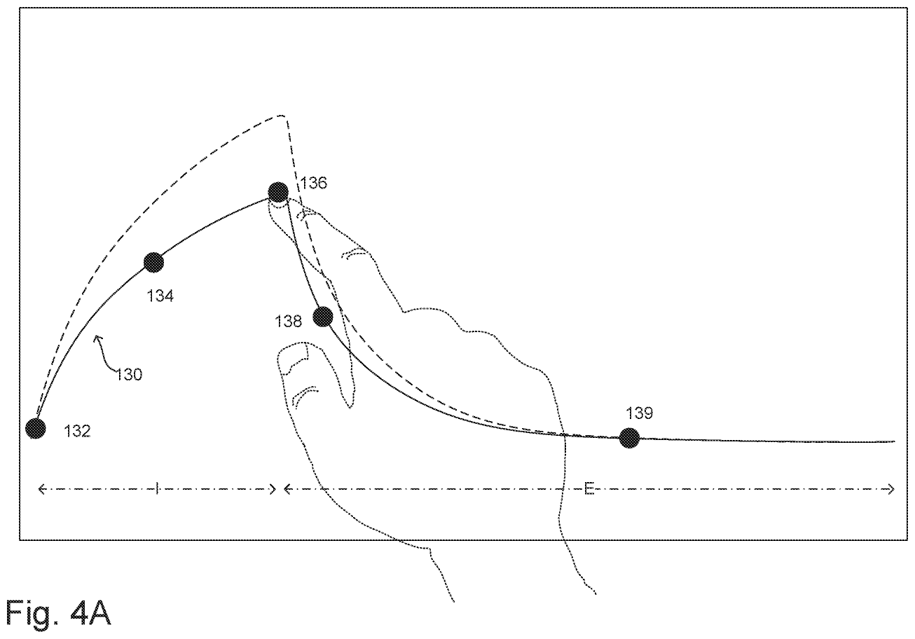

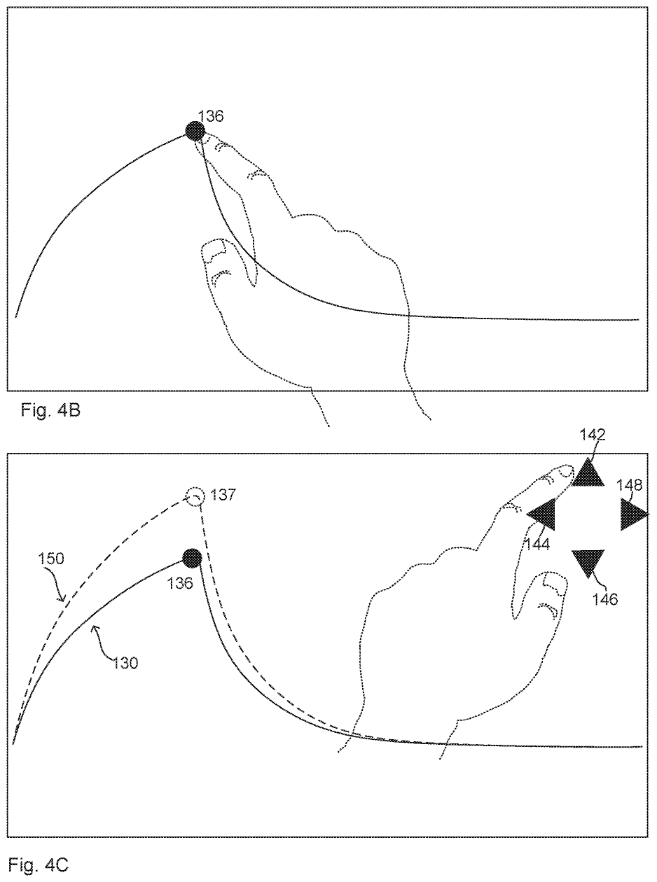

For example, referring to FIG. 4A, user interface 116 may display to the user the target pressure waveform 130 with one or more visual features 132, 134, 136, 138 and 139 corresponding to different waveform parameters. Each visual feature may be a point, or other visual icon, on or displayed in association with the target pressure waveform. For example, the visual feature 132 may correspond to one or more parameters related to the IPTT. The visual feature 134 may correspond to one or more parameters related to the inspiratory pressure shape. The visual feature 136 may correspond to one or more parameters related to the peak inspiratory pressure and/or the EPTT. The visual feature 138 may correspond to one or more parameters related to the expiratory pressure shape. The visual feature 139 may correspond to one or more parameters related to the peak expiratory pressure.

The user may activate, such as by manually adjusting, the one or more visual features for making changes to (e.g., values of) the related waveform parameters. For example, an adjustment to a visual feature may correspond to an adjustment to the related or corresponding therapy control parameter(s). Such a change may be made without requiring the user to perceive or understand the values associated with the waveform parameter changes.

For example, in the case of a touch screen, the user 102 may activate changes to a parameter by touching a corresponding visual feature on the target pressure waveform 130. Thus, the processor(s) 110 may detect such activation and/or an adjustment to a visual feature through a touch gesture on the touch screen. In one example, once the user touches a visual feature, such as the visual feature 136 as shown in FIG. 4B, one or more optional icons or arrows 142-148 may appear on the graphical user interface as shown in FIG. 4C. The user may adjust the visual feature (and thereby its corresponding parameter(s)) by touching any one of the icons or arrows 142-148. The arrows 142-148 may increase or decrease one or more parameter values, which may be presented by a change in the visualization of the target pressure waveform (e.g., show a change in shape).

With reference to the visual feature 136 shown in FIG. 4C, the user may change the position of the visual feature 136, and/or its corresponding parameter value, by touching any of the arrows 142-148. The user may adjust the time of the peak inspiratory pressure by touching the arrows 144 and/or 148. The forward arrow 144 may move the visual feature 136 towards the beginning of inspiration, which, in turn, may reduce the inspiratory pressure rise time. On the other hand, the backward arrow 148 may move the visual feature 136 towards expiration, which, in turn, may slow down the inspiratory pressure rise time.

By touching the arrows 142 and/or 146, the user may adjust the amplitude of the peak inspiratory pressure, or the amount of pressure applied at the peak time.

In another example, when the user 102 touches a visual feature, a menu may be displayed providing one or more options to adjust the visual feature or its corresponding parameter.

In yet another example, the user may adjust a visual feature and its corresponding parameter, by dragging or moving the visual feature with user contact of the visual feature on the touch screen, such as the visual feature 136, from its initial position to a new position 137 as shown in FIG. 4C. The corresponding parameter may be adjusted proportionally based on the new position 137 relative to the initial position. For example, if the new position 137 is lower than the initial position, then the corresponding parameter may be reduced proportionally. If the new position 137 is higher than the initial position, then the corresponding parameter may be increased proportionally.

In another aspect, in the absence of a touch screen or without relying on a touch screen, the user 102 may use a selector(s) 122 that is a menu selector to select any visual feature on the target pressure waveform 130 so as to adjust its corresponding parameter. For example, the user 102 may select the visual feature 136 of the target pressure waveform 130 to adjust the peak inspiratory pressure. When the visual feature 136 is selected, one or more icons or arrows 142-148 may appear on the graphical user interface. The user may adjust the visual feature 136 or its corresponding parameter value by using the menu selector(s) 122 to select any one of arrows 142-148.

In one embodiment, as shown in FIG. 4C, change in a visual feature or its corresponding parameter may lead to a change in the visualized shape or configuration of the target pressure waveform 130. As the user adjusts the visual feature or its corresponding parameter whether through the touch screen or the menu selector(s) 122, the graphical user interface may display any such change to the target pressure waveform 130. Optionally, the graphical user interface may display simultaneously the target pressure waveform 130 in its original configuration as shown by a solid curve, and its adjusted shape or configuration as shown by a dashed curve 150. In a still further option, additional boundary curves may be displayed to show the limits associated with how far such manual adjustments may be made.

1.2.2 Manual Optimization Guided with Trained Person

The system 100 may further be configured to permit a trained person, i.e., a person other than the user, to manually adjust the waveform parameters of pressure waveform 130 to personalize the waveform to the user. A trained person may have greater knowledge than the user regarding how each waveform parameter is likely to affect the user's comfort if adjusted and how much each waveform parameter should be adjusted. The trained person may adjust the waveform parameters in the same manner as described above using user interface 116. Alternatively, as will be described in greater detail below, system 100 (via network interface 118) may be configured to receive one or more commands or inputs (e.g., in communication signals) from wireless or external device 170 used by the trained person. The one or more commands or inputs received are used by system 100 to adjust the waveform parameters of target pressure waveform 130.

As part of the waveform personalization, the trained person may administer a series of A-B tests to the user with the user selecting their preferences for a first pressure waveform A or a second pressure waveform B at the end of each round of tests.

For example, the trained person may control system 100 to provide a pressurized flow of breathable gas to the airway of the user 102 in accordance with a first waveform A having a first set of waveform parameters for a first period of time. The first waveform A may be the default waveform 130 stored in memory 114 or may be a waveform with waveform parameters that have been adjusted by the trained person. Then the trained person may control system 100, via a user interface, to provide a pressured flow of breathable gas to the airway of the user 102 in accordance with a second waveform B having a second set of waveform parameters for a second period of time. At least one waveform parameter in the second set of parameters is adjusted relative to a same parameter in the first set of parameters. For example, the inspiratory waveform shape in the first set of parameters may be linear and the inspiratory waveform shape in the second set of parameters may be rounded or curved. Prior to providing the second waveform B to the user, the trained person may use user interface 116 to set the waveform parameters of the second waveform B. The second waveform B may be generated by adjusting the first waveform A.

After a first round of testing where the user has breathed under the first waveform A and the second waveform B, the trained person will ask the user which waveform they prefer. Additionally, the trained person may listen to any comments the user may have and/or ask the user one or more pointed questions with respect to how the user felt while breathing when the first waveform A was delivered and the second waveform B was delivered. For example, the user may indicate, or the trained person may ask the user to indicate whether, whether the user felt like they had enough fresh air when each waveform was delivered (e.g., meaning pressure was too low) or whether the user felt overinflated (e.g., meaning tidal volume was too high and/or EPTT is set too high (indicating late timing)).

Based on the user's indicated preference for first waveform A or second waveform B and the user's comments/responses, the trained person will perform another testing round providing the user with a first waveform (i.e., whichever of waveform A/B the user indicated a preference for) and a second waveform. The second waveform may be a waveform that the trained person has further made adjustments to (by adjusting one or more waveform parameters). For example, the second waveform may be an adjusted version of waveform A/B. At least one waveform parameter of the second waveform will have a different value or be adjusted relative to a same at least one waveform parameter of the first waveform. The trained person will again listen to the user's preference for the first or second waveform and any additional comments the user may have and based on the user's preference and comments, and the trained person may again adjust one or more waveform parameter settings to the first or second waveform and perform another A/B testing round. This process of continued A/B tests with replacement of one of the two waveforms with a further adjusted waveform after each testing round is continued until a waveform with the most comfortable combination of waveform parameter values is identified. Alternatively, the process may continue for a predetermined time or until the user indicates an acceptable level of comfort has been reached.

After the most (or an acceptably) comfortable waveform is identified, the waveform and the set of waveform parameters and their values are saved to memory 114.

### 1.2.3 System Guided Self-Optimization

In another aspect of the present technology, system 100 may be configured to further automate the above-described A/B waveform personalization such that system 100 automatically guides the user through a testing process with one or more waveform comparisons (e.g., A/B) or testing queries for identifying a comfortable pressure waveform. As such, the controller may be configured to deliver two or more different waveforms (e.g., A/B waves) where one or more parameters of each waveform differs. The user, such as by entry of input to the user interface of the system 100, may identify which one is more comfortable than the other(s) and thereby the one or more parameters associated with the selected waveform may become part of the comfort waveform for the user's further use with the therapy device. In such a process, the selection of the waveform by comparison effectively selects parameter(s) and the user does not even need to know or understand what parameters are changed since the selection is essentially based on the user's feeling of the delivered waveform. One or more of such comparisons may be implemented to further identify further parameters that may then also be combined with earlier parameters into the user's personalized comfort waveform.

For example, in this aspect, system 100 may include a selectable setting, e.g., within the graphical user interface presented to the user, to activate a waveform selection control loop during which system 100 will perform automatically guided waveform comparison testing to identify a personalized pressure waveform that is comfortable to the user. The processor(s) 110 may be configured to automatically perform a series of A/B tests with the user selecting their preference for waveform A or waveform B during each test run or loop. The settings or values of one or more parameters can be changed by processor(s) 110 with each A/B test. The A/B tests may continue in an automated manner until a comfortable (or the most comfortable) combination of waveform parameter values or settings is identified. The instructions for executing the waveform selection control loop may be stored in memory 114 and executed by processor(s) 110.

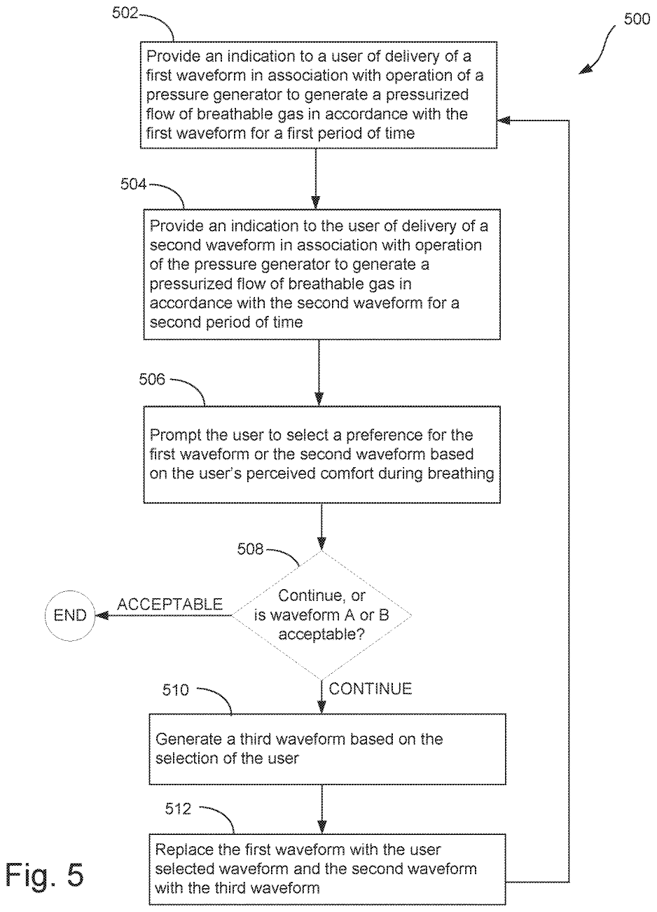

Referring to FIG. 5, a flow diagram of an automated waveform selection control loop is shown in accordance with the present technology.

In step 502, processor(s) 110 provide an indication to the user of a first waveform. For example, in one aspect, processor(s) 110 may control user interface 116 to display a message to the user that a pressurized flow of breathable gas is or will be delivered to the airway of the user in accordance with a first waveform. In association with providing the indication to the user, processor(s) 110 are also configured to operate the pressure generator 112 to generate a pressured flow of breathable gas in accordance with the first waveform for a first period of time. The first period of time is selected to permit the user to take several breaths and to appreciate the different aspects of the first waveform and the user's comfort regarding the same.

After the first period of time, in step 504, processor(s) 110 provide an indication to the user of a second waveform. For example, in one aspect, processor(s) 110 may control user interface 116 to display a message to the user that a pressurized flow of breathable gas is or will be delivered to the airway of the user in accordance with a second waveform. In association with providing the indication to the user, processor(s) 110 are also configured to operate the pressure generator 112 to generate a pressurized flow of breathable gas in accordance with the second waveform for a second period of time that occurs later than the first period of time. The second period of time may be of the same duration as the first period of time and is selected to permit the user to take several breaths and to appreciate, by feeling the delivered waveform, the different aspects of the second waveform and the user's comfort regarding the same.

The first and second waveforms are selected or generated by processor(s) 110 such that at least one waveform parameter of the second waveform has a different value or setting relative to a same at least one parameter of the first waveform. For example, the first waveform may have a linear or square inspiratory or expiratory shape and the second waveform may have a rounded inspiratory or expiratory shape. As another example, the first waveform may have a first peak inspiratory pressure and the second waveform may have a second peak inspiratory pressure that is different in value than the first peak inspiratory pressure. Processor(s) 110 may be configured to select the first and second waveform from a plurality of waveforms stored in memory 114 or in a database accessible by processor(s) 110 via network interface 118. Alternatively, processor(s) 110 may generate the first and second waveforms by making adjustment(s) to one or more waveform parameters of a predetermined waveform stored in memory 114.

In step 506, processor(s) 110 are configured to prompt the user (e.g., via user interface 116 or an external device 170) to select (via user input to interface 116 or to external device 170) a preference for the first waveform or the second waveform based on the user's perceived comfort during breathing. In some aspects, processor(s) 110 may further prompt the user for additional feedback regarding each of the first and second waveform. Such feedback may, for example, be in relation to whether the user felt overinflated or if the user felt like they were struggling to breathe or whether inspiration or expiration specifically felt difficult to the user. In some aspects, processor(s) 110 provide the user with the option of repeating steps 502, 504 if the user feels they need additional testing of the first and second waveform to form an opinion regarding their preference and to provide feedback.

In step 510, processor(s) 110 are configured to generate a third waveform, such as for comparison with another waveform (e.g., the first waveform, second waveform, or a fourth waveform), based at least on the selection of the user regarding the user's preference for the first waveform or the second waveform. The processor(s) 110 may further adjust the generation of the third waveform based on other feedback, such as subjective input, provided by the user in response to prompts by the processor (e.g., regarding whether the user felt overinflated, struggled to breath, etc.) and/or based on the outputs of one or more sensors 124. The processor(s) 110 may generate the third waveform by adjusting at least one waveform parameter of the first or second waveform (whichever was selected by the user in step 506). In one aspect, in performing step 510, processor(s) 110 are configured to generate a third waveform that has a set of parameters including at least one parameter that has a different value or setting than a same parameter of the first waveform and the second waveform. In other words, the third waveform has a different set of parameters values or settings relative the parameter values or settings of the first and second waveform.

In step 512, processor(s) 110 are configured to prepare for performing the control loop again by replacing the first waveform used in the cycle of the control loop with the waveform selected by the user between the first waveform and the second waveform (if necessary) and by replacing the second waveform with the generated third waveform. Then, processor(s) 110 are configured to perform the loop again starting from step 502.

In one aspect, the method 500 may further include step 508 for exiting the control loop and selecting a waveform that is at an acceptable level of comfort to the user. Step 508 may be performed on the first loop or cycle of method 500 or may be performed for the first time on the second or a subsequent loop or cycle of method 500. In step 508, processor(s) 110 may prompt the user (e.g., via user interface 116 or an external device 170) to indicate if the first waveform (A) or the second waveform (B) is at an acceptable comfort level to the user and the user wants to stop the A/B testing process. If the user indicates that neither the first nor the second waveform is at an acceptable level of comfort to the user, processor(s) 110 may continue the method 500 in step 510. Alternatively, if the user indicates that the first waveform or the second waveform is at an acceptable level of comfort to the user, processor(s) 110 are configured to exit the control loop and the method 500 ends. Processor(s) 110 may then store the waveform indicated as comfortable by the user in step 508 in memory 114.

In one aspect, the method 500 may be limited by processor(s) 110 to performance for a predetermined period of time or for a predetermined number of control loop cycles, as will be described in greater detail below.

### 1.2.4 Automated Optimization

System 100 may be further configured to perform waveform personalization in fully automated manner such as without requiring direct user input on a user interface.

### 1.2.4.1 Automated Waveform Adjustment Control Loop

In one aspect of the present technology, the processor(s) 110 of the system 100 may be configured to make changes or adjustments to the pressure waveform in an automated manner (e.g., without user input entered on a user interface) based on the outputs of one or more sensors 124 to personalize the pressure waveform and increase user comfort. The adjustments may be made as part of a control loop that runs repeatedly such as continuously or at predetermined times, as described in greater detail below.

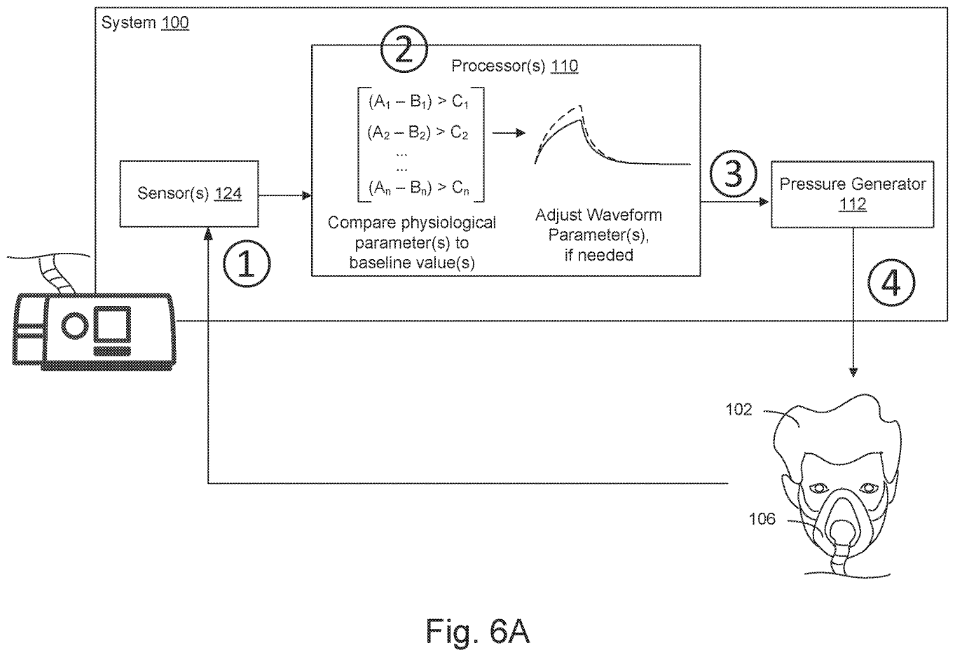

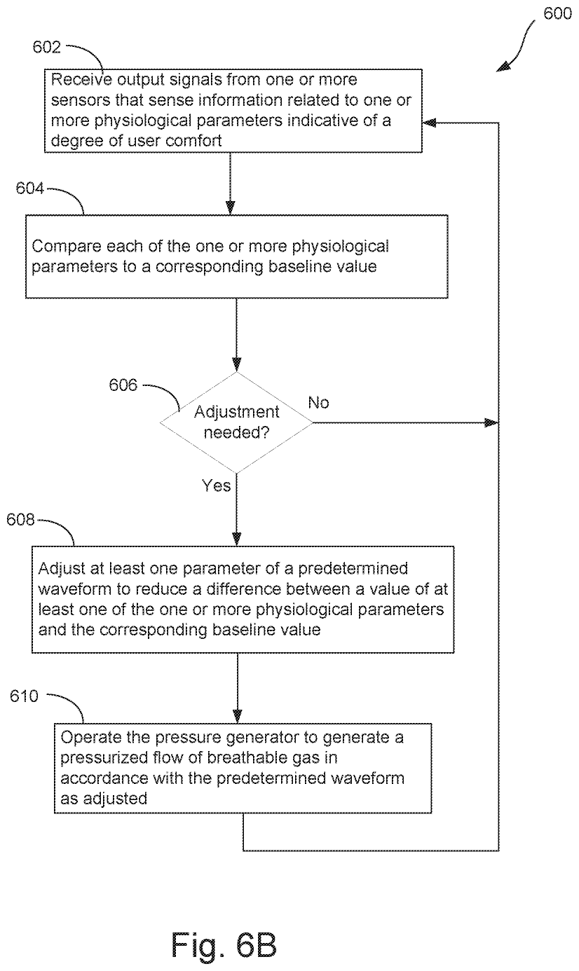

For example, referring to FIG. 6A, an exemplary waveform adjustment control loop implemented using system 100 is shown in accordance with the present technology.

In a first step, the processor(s) 110 may be configured to receive the output signals of one or more sensors 124 while a pressurized flow of gas generated by generator 112 is being delivered to the user 102 by system 100. The pressurized flow of breathable gas is delivered in accordance with a predetermined or target waveform, such as the waveform 130 illustrated in FIG. 1C or another default waveform. The predetermined or target waveform may be a waveform stored in memory 114 for providing therapy to user 102.

In some such implementations, sensors 124 are configured to generate output signals conveying information related to one or more physiological parameters that are indicative of the degree of the user's comfort with the waveform. The one or more physiological parameters may be sensed directly by sensors 124 or may be determined by processor(s) 110 based on the information from the output signals of sensors 124. The physiological parameters of the user 102 may include, but are not limited to, flow rate, pressure, CO₂, breathing rate, breathing effort, heart rate, tidal volume, respiration volume and/or the movement of the user. The sensors 124 may include a flow sensor for sensing flow rate, a pressure sensor for measuring pressure, a CO₂ sensor for sensing CO₂ concentrations (in the patient interface 106 or conduit 104), a heart rate sensor or monitor (e.g., a sensor for obtaining an electrocardiogram or a sensor for obtaining a photoplethysmogram), an accelerometer for measuring user movement (e.g., to detect user breathing waveform or comfort levels), or any other suitable sensors as needed for obtaining information related to the physiological parameters.

In a second step the control loop of FIG. 6A, based on one or more outputs of sensors 124, processor(s) 110 are configured to determine if user comfort is not at (e.g., below) an acceptable level. If processor(s) 110 determine, based on the outputs of sensors 124, the user's comfort is not at (e.g., below) an acceptable level, then in a third step of FIG. 6A, the processor(s) 110 may be configured to adjust at least one parameter (or more) of the predetermined waveform currently being used for improving user comfort. In a fourth step of FIG. 6A, processor(s) 110 operate the pressure generator 112 to generate a pressurized flow of breathable gas that is delivered to user 102 in accordance with the predetermined waveform as adjusted. Alternatively, if processor(s) 110 determine at the second step that the user comfort is at an acceptable level while the pressurized gas is being delivered to the user, then processor(s) 110 can refrain from making a comfort adjustment at the third step and the process will proceed to the fourth step.

Processor(s) 110 may perform the first, second, third, and fourth steps of the control loop of FIG. 6A repeatedly such as continuously (or iteratively) adjusting the predetermined waveform as needed based on the user's comfort level. Alternatively, as described in greater detail below, processor(s) 110 may perform the first, second, third, and fourth steps periodically as part of a staging process.

In one aspect, to determine the level of user comfort (or discomfort) as well as the adjustment(s) to be made to the predetermined waveform, in the second step of FIG. 6A processor(s) 110 are configured to compare values of the physiological parameters (derived from the outputs of one or more of sensors 124) to corresponding baseline values for each physiological parameter (e.g., respective baseline values for flow, pressure, breathing effort, heart rate, etc.). The baseline values may be values associated with each physiological parameter that indicate that the user is comfortable. Determination of the baseline values are described in greater detail below.

In FIG. 6A, the values for the distinct physiological parameters are represented as A₁, A₂, etc., to A₁ and the distinct baseline values corresponding to the physiological parameters are represented as B₁, B₂, etc., to Bₙ. For example, A₁ is a first physiological parameter (e.g., breathing rate) that is compared to a first baseline value B₁ (e.g., a value of the breathing rate that indicates the user is comfortable), A₂ is a second physiological parameter (e.g., tidal volume) that is compared to a second baseline value B₂ (e.g., a value of the tidal volume that indicates the user is comfortable), etc. It is to be appreciated that the control loop of FIG. 6A may employ any number of physiological parameters, any of which may be compared to a unique baseline value corresponding to it.

In some implementations, the comparison for each physiological parameter (A₁) may, for example, include:
(a) a calculation of a difference between a value of a physiological parameter (Aₙ) and a baseline value (Bₙ) for the physiological parameter, represented as (Aₙ−Bₙ) in FIG. 6A, and
(b) a determination of whether the difference (Aₙ−Bₙ) is greater (or less) than a predetermined threshold (represented as Cₙ in FIG. 6A) associated with the specific physiological parameter Aₙ being evaluated.

In one aspect, to adjust the waveform, processor(s) 110 may perform the comparison and adjustment based on evaluation of a single physiological parameter. In this aspect, if processor(s) 110 determine that the difference between the value of the physiological parameter is greater than (or less than) the predetermined threshold associated with the physiological parameter, processor(s) 110 may determine that the user comfort is below an acceptable level and a comfort adjustment to the predetermined waveform is needed. If this condition is not satisfied, processor(s) 110 determine that the user comfort is acceptable and no adjustment is needed.

In another aspect, processor(s) 110 may perform a comparison for each of a plurality of physiological parameters (e.g., two or more physiological parameters) to respective baseline values. This is represented in FIG. 6A as (A₁−B₁)>C₁, (A₂−B₂)>C₂, etc., to (Aₙ−Bₙ)>Cₙ. In one example of this aspect, if processor(s) 110 determine, based on the comparison, that the difference between any physiological parameter and its baseline value is greater than the predetermined threshold, processor(s) 110 determine that the user comfort is below an acceptable level and an adjustment to the predetermined waveform is needed. If this condition is not satisfied, processor(s) 110 determine that the user comfort is acceptable and no adjustment is needed. In another example of this aspect, processor(s) 110 may be configured to require determination that multiple (or all) of the plurality of physiological parameters differ from their respective baseline values by an amount that is greater than the respective predetermined thresholds to determine that user comfort is below an acceptable level.

It is to be appreciated that, in any of the aspects above, as an alternative to calculating a difference between the physiological parameter and the predetermined baseline value, a ratio between the value of each physiological parameter and a baseline value for the physiological parameter may be calculated. Each ratio may be compared to a corresponding predetermined threshold to determine if user comfort is at an acceptable level or if adjustment to the predetermined threshold is needed.

The baseline values may be determined in any of several different ways. For example, in one aspect, during a set-up mode of system 100 and without any therapy being provided to the user, values for sensors 124 may be recorded (e.g., in memory 114) to identify individualized baseline values indicative of the user being in a comfortable state. Alternatively or additionally, the baseline values may empirically be determined based on input attributes associated with the user, such as the height, weight, age, gender, activity level, medical conditions, etc. For example, baseline values for each physiological parameter indicated of user comfort may be identified for large numbers of users having different heights, weights, ages, genders, activity levels, medical conditions, etc. and stored in memory 114. Based on the specific user's attributes, which may be entered on a user interface, suitable baseline values may be determined or selected by processor(s) 110 such as where the values are associated with such attributes in the memory of the device.

It is to be appreciated that in any of the aspects described above, instead of baseline values, ranges of baseline values may be used. For example, for each physiological parameter, processor(s) 110 may be configured to determine whether the value for the physiological parameter is within a range of baseline values for that physiological parameter that are indicative of an acceptable level of user comfort. If one or more of the assessed physiological parameter values are not within the corresponding ranges, processor(s) 110 may determine that user comfort is below an acceptable level and adjustment to the predetermined waveform is needed. The ranges of baseline values for each physiological parameter may be identified in a similar manner as described above, i.e., by recording sensor outputs when the user is in a comfortable state and/or using physical attributes associated with the user.

It is to be appreciated that the techniques for identifying baseline values or ranges described herein are not meant to be limiting and that other methods for identifying baselines values or ranges for use with the present technology are contemplated to be within the scope of the present disclosure.

After processor(s) 110 determine that an adjustment to the predetermined waveform is needed based on the comparison of the physiological parameters to the baseline values or ranges of baselines values, processor(s) 110 are configured to adjust at least one waveform parameter of the predetermined waveform (e.g., any of the waveform parameters discussed above) to improve user comfort. The control loop shown in FIG. 6A may be performed iteratively. If processor(s) 110 determine based on the comparison of the physiological parameters to the baseline values or ranges that user comfort is at an acceptable level, processor(s) 110 will not make any adjustments to the waveform and the loop and will return to the beginning of the loop to again receive and monitor the data from sensor(s) 124.

In one aspect, the control loop may be performed by processor(s) 110 for a predetermined amount of time or for a predetermined number of control loops as part of a staging process, as described in greater detail below.

The adjustment to the at least one waveform parameter of the predetermined waveform is selected by the processor(s) 110 to reduce a difference between a value of at least one of the physiological parameters and the baseline value corresponding to the physiological parameter. It is to be appreciated that processor(s) 110 may be configured to adjust a single waveform parameter in each operation of control loop or multiple waveform parameters in each operation of control loop. Additionally, processor(s) 110 may give priority to adjustment of certain waveform parameters that are considered to have a greater relative impact on the user's comfort than other waveform parameters that are considered to have a lesser relative impact on the user's comfort. Prioritized waveform parameters will be adjusted first.

In some implementations, processor(s) 110 may be configured to select the amount or degree of adjustment (e.g., a relatively small adjustment or a large adjustment) to the one or more waveform parameters based on the amount of difference calculated by processor(s) 110 between the one or more physiological parameters and their respective baseline values during the comparison. Processor(s) 110 may be configured to make a larger adjustment to a waveform parameter if a larger difference is calculated or a smaller adjustment to a waveform parameter if a smaller difference is calculated.

Referring to FIG. 6B, a flow diagram of a method 600 for an automated waveform adjustment control loop is shown in accordance with the present technology. In step 602, processor(s) 110 receive output signals from one or more sensors that sense information related to one or more physiological parameters that are indicative of a degree of user comfort. The output signals are received during delivery of a pressurized flow of breathable gas to the airway of the user in accordance with a predetermined waveform. In step 604, processor(s) 110 compare each of the one or more physiological parameters to a corresponding baseline value. In step 606, processor(s) 110 determine, based on the comparison, if an adjustment to at least one waveform parameter of the predetermined waveform is needed to improve the comfort of the user. If processor(s) 110 determine that no adjustment is needed, the method 600 returns to step 602. Alternatively, if processor(s) 110 determine that an adjustment is needed, processor(s) 110 are configured to adjust the value or setting of at least one waveform parameter of the predetermined waveform. The adjustment is made to reduce a different between a value of at least one of the one or more physiological parameters and the corresponding baseline value for the at least one physiological parameter. For example, as described above, processor(s) 110 may adjust any one of an inspiratory shape, an expiratory shape, a rise time of an inspiration phase, a fall time of an expiration phase, an inspiratory pressure trigger threshold, an expiratory pressure trigger threshold, a peak inspiratory pressure, a peak expiratory pressure, an inspiratory pressure trigger threshold, and an expiratory pressure trigger threshold of the predetermined waveform. In step 610, processor(s) 110 operate the pressure generator to generate a pressurized flow of breathable gas in accordance with the predetermined waveform as adjusted in step 608 and then the method returns to step 602.

The method 600 and the features described above may be activated by user input to user interface 116 or from one or more commands received by system 100 from an external device, such as device 170.

### 1.2.4.2 Automated Personalized Waveform Approximation

In another aspect of the present technology, the processor(s) 110 of the system 100 may be configured to approximate a user's breathing pattern using sensor outputs from sensor(s) 124, or values calculated therefrom. The processor(s) 110 may then generate a personalized pressure waveform based on the approximation. In this way, the personalized waveform approximately mirror's the user's natural breathing pattern such that the user's comfort during delivery of a pressurized flow of breathable gas in accordance with the personalized waveform is maximized.

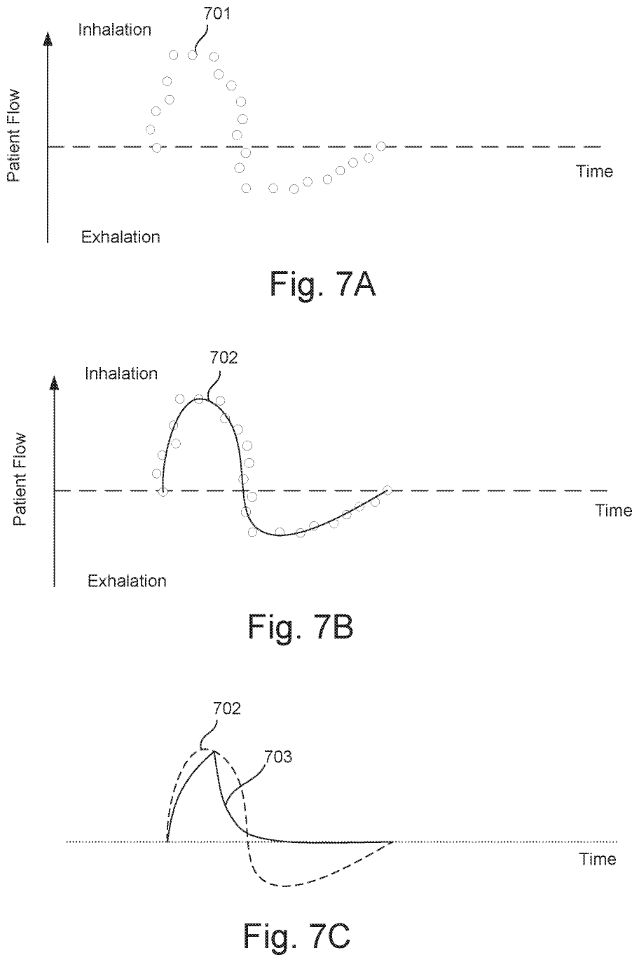

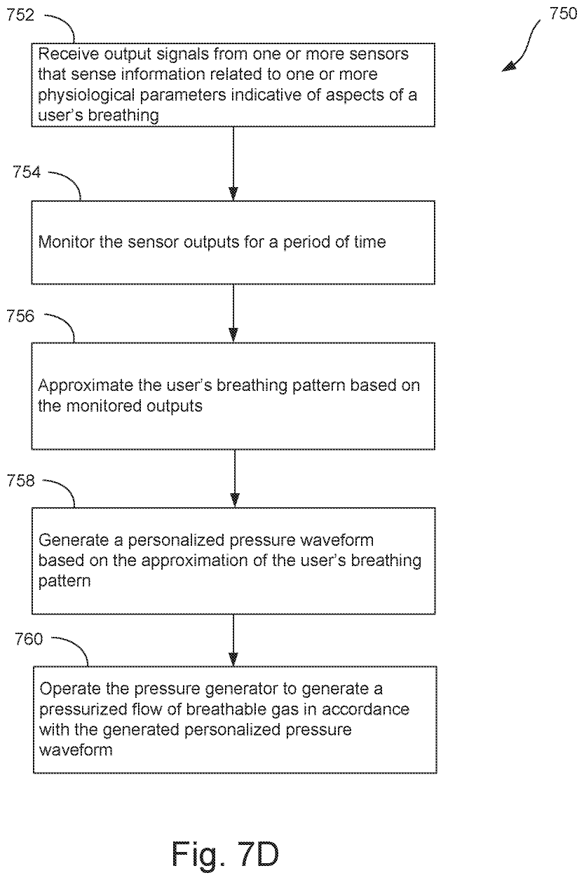

For example, FIGS. 7A-7C illustrate an exemplary waveform personalization based on approximation of the user's breathing pattern in accordance with aspects of the present technology. Processor(s) 110 receive output signals from one or more sensor(s) 124 that sense information related to one on or more physiological parameters indicative of aspects of a user's breathing, such as when a user is not receiving respiratory pressure therapy or not wearing a respiratory interface (e.g., mask). The physiological parameters are parameters that may be used to approximate the user's breathing pattern. For example, the physiological parameters may include, but are not limited to, one or more of flow rate, pressure, CO₂, movement of the lungs, and/or bioimpedance of the lungs. In one aspect, sensor(s) 124 may include one or more of:
(a) a flow sensor for sensing flow rate,
(b) a pressure sensor for measuring pressure,
(c) a CO₂ sensor for measuring the concentration of CO₂ in, for example, the patient interface 106, conduit 104, or another portion of the air delivery path or airway path,
(d) an accelerometer (e.g., coupled to the user's chest with chest bands or a noncontact sensor) for measuring the lung movement of the user,
(e) one or more EEG patch sensors, e.g., that can be adhered to the user's chest, for sensing bioimpedance of the user's lungs which may change with different respiration volumes, or
(f) any other suitable sensors as needed for obtaining information related to the physiological parameters.

Processor(s) 110 monitor the sensor signal outputs of sensor(s) 124 for a period of time and may store the outputs in memory 114. FIG. 7A shows a portion of the data points 701 from a flow sensor plotted over time. The data points 701 may be direct outputs from a flow sensor (of sensor(s) 124) or data points derived therefrom by processor(s) 110. The portion of data points 701 shown in FIG. 7A are the points corresponding to a breathing cycle (including inhalation and exhalation) of the user. In one aspect, processor(s) 110 are configured to partition the monitored output data into breathing cycles and use the partitioned data belonging to at least one of the breathing cycles in the waveform personalization process of the present technology.

The data points 701 as plotted roughly correspond to the user's natural breathing pattern. As shown in FIG. 7B, processor(s) 110 are then configured to approximate the user's breathing pattern based on the data points 701. The approximated breathing pattern is represented by the curve 702. In one aspect, processor(s) 110 generate or determine the curve 702 by processing data points 701 and determining a best fit curve that fits to the data points 701 e.g., within an acceptable level of accuracy. In identifying the best fit curve 702, processor(s) 110 may determine that some of the data points 701 are outliers and exclude these data points from the analysis and the curve 702.

In one aspect, the processor(s) 110 may be configured to approximate the user's breathing pattern by averaging data from a plurality of breathing cycles. For example, processor(s) 110 are configured partition data points 701 into a series of breathing cycles and determine curves 702 (e.g., by identifying the best fit line or curve in each) for the data points 701 in each of the series of breathing cycles. Processor(s) 110 may then generate a curve 702 that is the average of all of the determined curves 702 of the series of breathing cycles for the monitored period of time. The average curve 702 is the approximation of the user's breathing pattern. This approach may smooth out any irregularities in the data that may exist in an individual breathing cycle, but do not exist across the average of the breathing cycles.

In any of these aspects for generating the approximation of the user's breathing pattern 702, processor(s) 110 are then configured to generate a personalized pressure waveform 703 based on the approximation of the user's breathing pattern 702, as shown in FIG. 7C. For example, processor(s) 110 may extract breathing parameters from the approximated breathing waveform 702. Based on the extracted breathing parameters, processor(s) 110 may determine pressure waveform parameters. From the calculated pressure waveform parameters, processor(s) 110 may generate the personalized pressure waveform 703. The processor(s) 110 may then store the personalized pressure waveform 703 in memory 114 and/or operate the pressure generator 112 to generate a pressured flow of breathable gas in accordance with the personalized pressure waveform 703.

Referring to FIG. 7D, a flow diagram of a method 750 for generating a pressure waveform that is personalized to the user based on an approximation of a user's breathing pattern is shown in accordance with the present technology.

In step 752, processor(s) 110 receive output signals from one or more sensors that sense information related to one or more physiological parameters indicative of aspects of a user's breathing. The one or more physiological parameters may be any of the physiological parameters described above. The physiological parameters may be sensed directly by one or more sensor(s) 124 or calculated by processor(s) 110 based on the outputs of sensor(s) 124. In step 754, processor(s) 110 monitor the sensor outputs for a period of time. The period of time may be selected to capture data related to one or a plurality of breathing cycles of the user. In step 756, processor(s) 110 approximate the user's breathing pattern based on the monitored outputs. In step 758, processor(s) 110 generate a personalized pressure waveform based on the approximation of the user's breathing pattern. In step 760, processor(s) 110 operate the pressure generator 112 to generate a pressurized flow of breathable gas in accordance with the generated personalized pressure waveform.

The method 750 and the features described above may be activated by user input to user interface 116 or from one or more commands received by system 100 from an external device, such as device 170.

It is to be appreciated that in any of the aspects described herein, processor(s) 110 may use the physiological parameters sensed or calculated from sensor(s) 124 to personalize the user's experience in ways other than the waveform parameters of a pressure waveform. For example, CO₂ determined from or sensed from the outputs of sensor(s) 124 may be used by processor(s) 110 to control (and optimize) vent flow rate(s) of the flow of gas through one or more vents of system 100. For example, system 100 may include one or more vents in patient interface 106 and/or conduit 104 to allow gas in the air flow delivery path (include conduit 104 and patient interface 106) to escape or be vented out to atmosphere. The CO₂ concentrations in the patient interface 106 and/or conduit 104 may indicate if there is a sufficient level of washout in patient interface 106 or if vent flow needs to be increased (e.g., by opening the vent(s) or increasing the vent opening size) so that the user 102 is not rebreathing expired CO₂ that is still in patient interface 106 and/or conduit 104.

### 1.2.5 Staged Optimization

In any of the waveform personalization aspect described herein, the personalization may occur in one or more stages of predetermined time. In this regard, optimizing a pressure waveform to the user's comfort may be time consuming (e.g., due to extensive experimenting and adjustments) and/or computationally intensive for processor(s) 110. However, a user's comfort may be significantly improved by performing the waveform personalization in stages without necessarily finding the “optimal” waveform in one session. For example, rather than achieving the most comfortable combination of settings or waveform parameters for a pressure waveform in one session, a shorter optimization session may be conducted initially for a predetermined period of time (e.g., 5 minutes, 10 minutes, 30 minutes, 45 minutes, 1 hour, etc.). Then, the user may continue to use the system 100 as normal to receive therapy.

In one aspect, processor(s) 110 are configured to prompt the user when an optimization session has finished and further prompt the user (e.g., after a few days) at a later time to perform a further optimization session to make additional adjustments to the pressure waveform to improve comfort. Processor(s) 110 may prompt the user by outputting a notification to the user interface 116 (e.g., a display message or other visual prompt or an audio prompt) to indicate an optimization session is over or to perform a further optimization session. Alternatively or additionally, processor(s) 110 may transmit prompts to an external device or application (e.g., device 170) via network interface 118.

In some aspects, processor(s) 110 are configured to limit the optimization session to a predetermined time period (e.g., 5 minutes, 10 minutes, 30 minutes, 45 minutes, 1 hour, etc.) or to a predetermined number of control loops (e.g., 5 control loops, 10 control loops, 100 control loops, etc.). For example, processor(s) 110 may be configured to impose these limits with respect to any of the waveform personalization processes described in sections 1.2.3 and 1.2.4.

Moreover, processor(s) 110 may be configured to prioritize adjustment of one or more waveform parameters in earlier stages or sessions that have a larger impact on user comfort. This may help limit the optimization time in each stage as well as decrease the time it takes to identify a comfortable waveform for the user. For example, processor(s) 110 may be configured to adjust (or prompt the user to adjust) the waveform shape (inspiratory and/or expiratory shape) first in a first stage before attempting to adjust other waveform parameters. The waveform shape may be the only parameter (or parameters) adjusted in the first stage. The waveform shape (e.g., linear vs. curved) may have the most impact on user comfort. After the waveform shape is sufficiently adjusted to the user's comfort, processor(s) 110 may be configured to assign a descending level of priority to other waveform parameters based on the impact they are likely to have on user comfort. For example, processor(s) 110 may be configured to adjust (or prompt the user to adjust) the expiratory pressure relief (EPR level) of the waveform in a second stage (or after waveform shape adjustment is attempted or completed) as the amount of expiratory pressure relief may have the second most impact on the user comfort.

### 1.2.6 Optimization Efficiencies Based on Additional Data

Further efficiencies may be realized in any of the waveform personalization aspects described herein by using additional user data to improve the personalization process. For example, user data (e.g., gender, weight, height, age, fitness level, etc.) may be input to system 100 (e.g., by User Input or by Data Received Through Network Interface 118) and Used by processor(s) 110 to narrow the settings or waveform parameters presented or limit the number of options to the user or that are used in the any of the control loops described above.

In one example, based on the user data, processor(s) 110 may select one or more waveforms from a plurality of waveforms stored in memory 114 or an external database accessible by processor(s) 110 via network interface 118. Each of the plurality of waveforms has a set of waveform parameters that have been preset and each waveform has a different combination of parameter values or settings for their set of waveform parameters. The plurality of waveforms may have previously been tested or determined from large populations of users to determine and associate to the waveforms the types of users that found each of the waveforms most comfortable. It is to be appreciated that any of the plurality of waveforms stored in memory 114 with preset combinations of settings may be selectable by the user 102 through interface 116 for use with system 100 for delivery of a pressurized flow of breathable gas in accordance with the selected waveform. Additionally, user 102 may save one or more custom waveforms (e.g., using any of the personalization aspects described herein) with preset settings or waveform parameters that may be selected directly by the user or used by processor(s) 110.

Based on the user data, processor(s) 110 may select one or more waveforms from the plurality of waveforms that the user, with the user's particular set of user data, is likely to find most comfortable. The selected waveforms may then be further personalized for example using any of the personalization implementations previously described such as to select adjustments to parameters of such waveforms. The selection of the waveforms based on the user's data may cut down on optimization time significantly since the initially selected waveforms may have a closer waveform parameter relationship to the waveform that the user will ultimately identify as the most comfortable using the personalization aspects described above. Thus, fewer waveform parameter adjustments will likely be needed to identify the waveform that is comfortable and personalized to the user.

The selected waveforms may be used in any of the personalization aspect described above. For example, if the user (or a trained professional) is to manually adjust the parameters of a pressure waveform, processor(s) 110 may select a waveform from the above-described plurality of waveforms that the user is likely to find comfortable as a starting point. The user (or trained professional) will then adjust the selected waveform. This may lead to fewer adjustments.

In another example, if the user selects the comparison testing (e.g. A/B testing) control loop described above in section 1.2.3, the processor(s) 110 may first select a first waveform A and a second waveform B from the plurality of stored and associated waveforms based on the user data. The processor(s) 110 may then use the selected waveforms A and B in the automated A/B testing process described in section 1.2.3. This may reduce the number of A/B tests required to identify a waveform with waveform parameter settings that are comfortable for the user.

In another example, if the user selects the control loop described above in section 1.2.4.1, the processor(s) 110 may select the predetermined waveform that is to be adjusted in the control loop from the plurality of stored waveforms based on the user data. The processor(s) 110 may then use the selected waveform in the control loop and make iterative adjustments based on the physiological parameters as described above. This may reduce the number of control loop iterations required to adjust the waveform to an acceptable level of comfort for the user.

In another example, if the user selects the process described above in section 1.2.4.2 for personalizing a waveform based on an approximation of the user's breathing pattern, the processor(s) 110 may factor into the generating step 758 a waveform selected from the plurality of stored waveforms based on the user data. The processor(s) 110 may select a waveform that is both (i) found to be comfortable by users that have similar user data (height, weight, age, etc.) to the user 102 and (ii) matching the approximated breathing pattern of user 102. This may lead to a pressure waveform that is personalized and even more likely to be comfortable to the user 102.

### 1.2.7 Optimization Constraints

In any of the waveform personalization aspect described herein, processor(s) 110 may be configured to impose constraints or limits on the adjustments made to the waveform parameters by the manual adjustments of the user or trained professional or the automated adjustments made by the processor(s) 110 to the pressure waveform. For example, processor(s) 110 may limit the adjustments to one or more of the waveform parameters to particular ranges that are safe to the user or within prescribed therapy requirements for the particular waveform parameters. These limitations may also be applied to the process described in FIG. 7 in step 758. In this way, the resulting personalized pressure waveform from any of the personalization aspects described herein will be safe and may comply with therapy prescriptions.

### 2. System Modes

The system 100 is configured to operate with various modes in accordance with the programming of its controller (e.g., comprising processor(s) 110). Such modes may include an operational mode and a set-up configuration mode. The set-up configuration mode may be an active-therapy user-feedback adjustment mode.

### 2.1 Operational Mode

During the operational mode, the system 100 may provide a therapy to the user according to parameters that are set up from the configuration mode. Such an operation mode may be a typical therapy mode during which time a patient receives therapy from the system 100. For example, in the case of a sleep disordered breathing therapy device, the operation mode would typically provide therapy during a sleeping session. Such a mode does not typically provide the user with the option of making manual adjustments to the therapy settings of the device.

In one aspect, the automated waveform adjustment control loop described above in relation to FIGS. 6A-6B and in Section 1.2.4.1 may be performed during the operational mode of system 100. In this aspect, system 100 will continuously and automatically adjust the waveform 130 while therapy is being provided to increase user comfort.

In some implementations, processor(s) 110 may be configured to determine the sleep state of the user while therapy is being provided to the user using system 100. Processor(s) 110 may be configured to activate (or allow activation of) the automated waveform adjustment control loop described above in relation to FIGS. 6A-6B and in Section 1.2.4.1 when the user is in certain sleep states and prevent activation of the waveform adjustment control loop when the user is in other sleep state(s). For example, when it is determined that the user is in an awake state or a light sleep state, the processor(s) 110 may activate the automated waveform adjustment control loop described above in relation to FIGS. 6A-6B and in Section 1.2.4.1. When it is determined that the user is in a sleep state, processor(s) 110 may deactivate the automated waveform adjustment control loop and return the waveform 130 to its default therapy prescribed parameters. Processor(s) 110 may be configured to determine the sleep state of the user 102 based on outputs from sensor(s) 124.

In any of the waveform personalization aspects described herein, when a personalized waveform process (such as described in sections 1.2.1-1.2.4) is performed and a personalized waveform that is comfortable to the user is identified, the personalized waveform may be stored in memory 114 and used during an operational mode of system 100 to deliver a pressured flow of breathable gas to the user in accordance with the personalized, comfortable waveform. In some aspects, multiple such personalized waveforms identified as comfortable to the user from a personalized waveform process (such as described in sections 1.2.1-1.2.4) may be stored in memory 114 and may be selected by the user for use in an operational mode or may be selected automatically by processor(s) 110 for use in the operational mode based on detection of different conditions (e.g., based on evaluation of data from sensor(s) 124 or other detected conditions). However, such a waveform might not be suitably therapeutic such as to satisfy a prescribed pressure or even be sufficient to avoid disordered breathing events. Thus, the processor(s) 110 may be configured to change the comfortable waveform to a more therapeutic waveform or morph the comfortable waveform to a more therapeutic version of the comfortable waveform (and vice versa) at certain times, such as after an initial period of time of use in a sleep session or upon detection of certain events such as sleep or sleep disordered breathing. For example, processor(s) 110 may be configured to operate the generator 112 to provide the personalized waveform initially or when it is determined that the user is in an awake or light sleep state and transition to a default therapy prescribed waveform (prior to adjustment) when the user is determined to be in a deep sleep state. Processor(s) 110 may use sensor(s) 124 to determine the sleep state or may use sleep history times to transition between the default therapy waveform and the personalized waveform.

Processor(s) 110 may be configured to control generator 112 to transition (or alternatively switch) between using different waveforms (e.g., previously learned waveforms) that are stored in memory, such as a first waveform and a second waveform, which may be based on one or more detected conditions (e.g., detected based on data from sensor(s) 124) and/or user input. In some aspects, the first waveform may be a default therapeutic waveform and the second waveform may be a personalized waveform (obtained using the waveform personalization processes described herein). In other aspects, the first waveform is a first personalized waveform and the second waveform is a second personalized waveform with at least one different waveform parameter value than a corresponding waveform parameter of the first waveform. In any of these aspects, processor(s) 110 may be configured to control generator 112 to transition or switch between the first waveform and the second waveform, such as based on the detected sleep state of the patient, as described above. Similarly, the processor(s) 110 may also be configured to present a user interface to allow the user, by user input, to select between the different waveforms, so that the generator 112 transitions from delivering the first waveform to the second waveform using, where the user can choose between different labeled characterizations of the different waveforms (e.g., one suitable for when the patient has congestion, cold or influenza (flu) or seasonal allergy (e.g., hayfever) and another suitable for when the patient is normal (e.g., no congestion)).

Processor(s) 110 may also be configured to control generator 112 to transition or switch between the first waveform and a second waveform based on detecting one or more conditions of the user in addition to, or instead of, the sleep state of the user. For example, processor(s) 110 may be configured to transition or switch between the first waveform or the second waveform based on detecting whether the user is congested. In one aspect, when the processor(s) 110 detects that the user is in a congested state, the processor(s) 110 may be configured to control generator 112 to transition or switch to the first waveform and when the processor(s) 110 detects that the user is in a non-congested state, the processor(s) 110 may be configured to control generator 112 to transition or switch to the second waveform. In this aspect, the first waveform may be a default therapeutic waveform or a personalized waveform that is appropriate for the user experiencing congestion. In the case where the first waveform is a personalized waveform, the first waveform may be a waveform previously identified and stored in memory by using any of the waveform personalization aspects described herein while the user is experiencing congestion. The second waveform may be a waveform previously identified and stored in memory by using any of the waveform personalization aspects described herein while the user is in a non-congested state. In some aspects, the second waveform is identified using the waveform personalization aspects described herein while the user is in a non-congested state and is in an awake state.

In one aspect, processor(s) 110 may be configured to detect whether the user is in a congested or non-congested state based on data from (or derived from) sensor(s) 124, and which may be further based on detection of the user being in an awake state. In one example, processor(s) 110 monitor one or more of the physiological parameters described herein using sensor(s) 124. The processor(s) 110 determine whether a current value or an average of a set of values of a current window or period of time (e.g., spanning one or several breaths from a current moment in time to a previous moment in time) of the monitored physiological parameter is different than a predetermined baseline value associated with the monitored physiological parameter. Alternatively, processor(s) 110 determine whether the current value or average is outside of (above or below) a predetermined baseline range of values of the monitored physiological parameter. In one aspect, the predetermined baseline value or range of values may be a known value or range that is expected from a user when the user is not experiencing congestion. In one aspect, the predetermined baseline value or range comprises, or is determined by processor(s) 110 based on, a historical set of values of the physiological parameter. In any of these aspects, if the monitored physiological parameter is determined by processor(s) 110 to currently be different than the predetermined baseline value by a predetermined amount or to be outside of (e.g., above or below) the predetermined range (e.g., the historical set of values), processor(s) 110 are configured to determine that the user is in a congested state. In some implementations, the processor(s) 110 may determine whether the user is in a congested state periodically (e.g., every 5 minutes, every hour, etc.). In some aspects, the physiological parameter monitored may be a shape volume and/or a breathing rate, which may be indicative of the flow profile of the user and differences from the user's norm may be deemed congestions. In some aspects, processor(s) 110 may be configured to monitor multiple physiological parameters and require that two or more physiological parameters indicate (based on comparison to the corresponding baseline values or ranges of each physiological parameter) that the user is congested to determine that the user is congested. In some implementations, congestion may be detected by detecting flow limitation, such as by detection of shape of the flow signal such as flow flattening or inspiratory flow flattening of the flow rate signal but when the patient is also in a detected wake state or non-sleep state, the flow limitation is deemed to be congestion.

In some aspects, rather than directly automatically transitioning or switching between the first waveform and the second waveform, when processor(s) 110 detect that the user is in a congested state, processor(s) 110 may be configured to alert or notify the user (e.g., via user interface 116) that congestion is detected and may then prompt the user on a user interface with a query asking whether the user would like to switch from the first waveform to the second waveform (e.g., a waveform suitable for congestion). The processor(s) 110 may then proceed with the transition based on input by the user that permits or confirms the switch.

In some aspects, processor(s) 110 are configured to switch or transition between the first waveform and the second waveform (or prompt the user to identify whether the user wants to switch or transition between the waveforms) based on whether the user is in a congested or non-congested state only when the processor(s) 110 detect that the user is in an awake state. In other aspects, regardless of the sleep or awake state of the user, processor(s) 110 are configured to switch or transition between the first waveform and the second waveform (or prompt the user) based on whether the user is in a congested or non-congested state.

In one aspect, the transitioning between the first waveform and the second waveform (or any number of waveforms) includes gradually adjusting the waveform parameter values or settings of a delivered waveform between (to or from) the first waveform (e.g., a therapy waveform) and the waveform parameter values or settings of the second waveform (e.g., a personalized waveform) and vice versa.

Moreover, processor(s) 110 may be configured to implement the waveform personalization aspects described herein with EPR and ramp features. For example, when the user is determined to be in the awake state and the ramp feature is activated, processor(s) 110 use the personalized waveform, but gradually increase the pressure levels using the protocols of the ramp features until the full extent of the pressure defined in the parameters in the personalized waveform is reached. The EPR feature may be activated during use of the personalized waveform but the pressure relief level may change based on sleep state.

### 2.2 Set-Up Configuration Mode

During the set-up configuration mode which may be an active-therapy user-feedback adjustment mode, the user or a trained professional may switch (e.g., via user interface 116 or 176) between previously stored waveforms (e.g., default waveforms or previously identified personalized waveforms) to identify a comfortable waveform. The previously stored waveforms may include waveforms having different shapes and waveform parameters, as described above. When a user selects a waveform from the stored waveforms, the processor(s) 110 may operate the pressure generator 112 to supply pressure according to the selected waveform. The user may repeat this process several times selecting different waveforms for delivery of pressure to identify a waveform that is comfortable to the user. As described in greater detail herein, selection of a waveform from the stored waveforms and other adjustments to the parameters of the waveforms may be made by the user on the user interface 176 of a wireless device 170 (e.g., a smartphone). In this way, the wireless device 170 may serve as a user interface and as a remote control of the system 100 by sending waveform selection control signals to the processor(s) 110 of the system for operation of the pressure generator 112. The wireless device 170 may be configured to also display a description or visual representation of each different waveform so as to differentiate each waveform that may be selected by the user.

Moreover, during the set-up configuration mode, the user or a trained professional may manually adjust (e.g., as described in Sections 1.2.1 and 1.2.2) one or more parameters for setting up therapy operations, such as the waveform parameters previously described for controlling therapy via the user interface 116. Moreover, during the active-therapy user-feedback adjustment mode, any one of the automated personalization processes described in Sections 1.2.3 and 1.2.4 may be activated to adjust one or more waveform parameters of the target waveform 130 to achieve a comfortable, personalized waveform. In any case, in the set-up mode, processor(s) 110 are configured to simulate therapy by operating generator 112 to provide a pressurized flow of breathable gas in accordance with the target waveform 130 to the airway of the user. During this mode, the waveform parameters of waveform 130 may be adjusted manually or automatically, which may be within predetermined or permissible constraints, and the processor(s) 110 may operate the pressure generator 112 such that the adjustments are implemented in real time or near real time and sensory feedback is provided to the user of the adjustments. In this way, the user will perceive the adjustments and can make further manual changes, instruct the trained professional to make further manual changes, and/or respond to user prompts regarding (e.g., in the assisted and automated A/B testing aspects) the user's perceived comfort in view of the waveform adjustments.

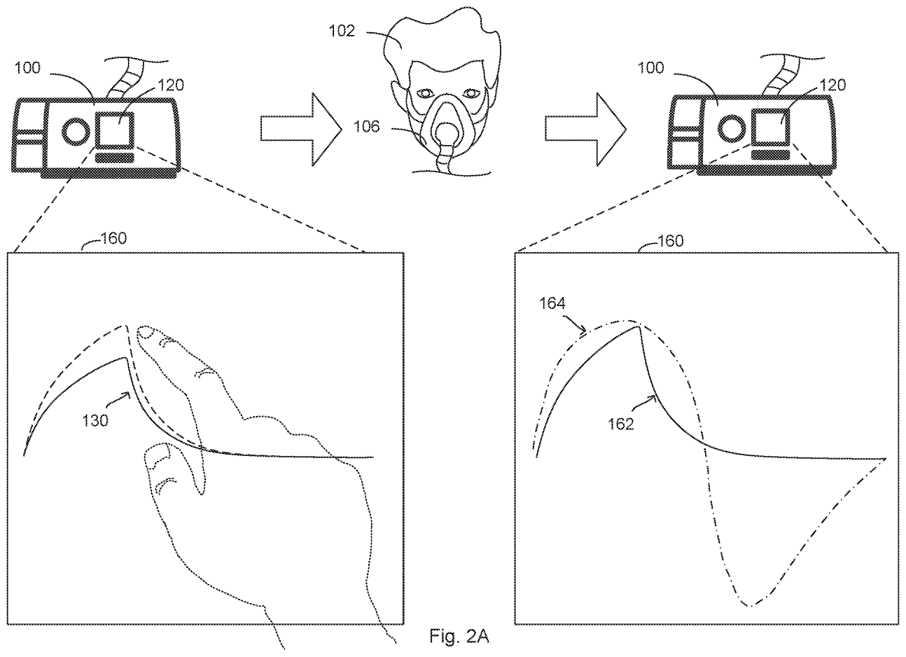

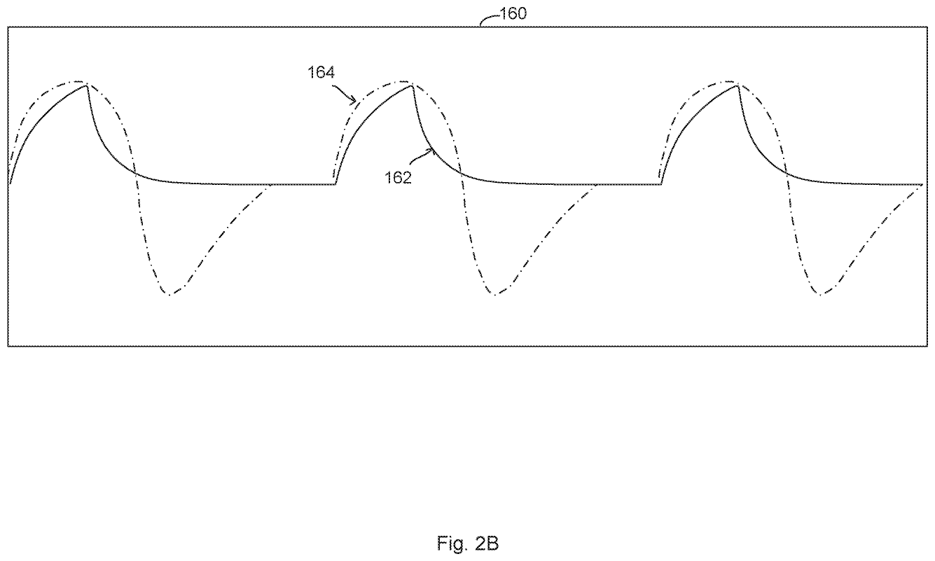

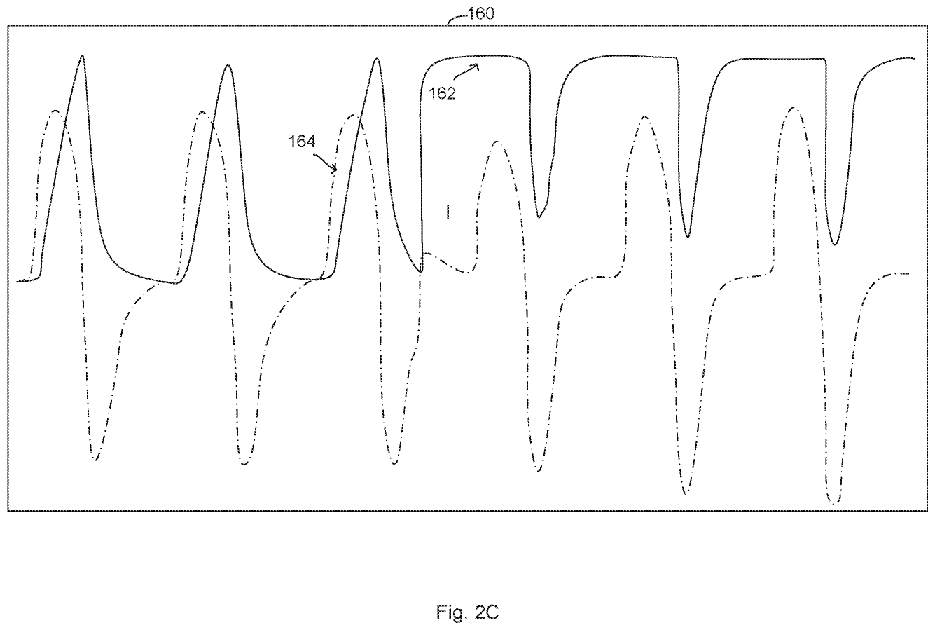

The user interface 116 may give the user control over making modifications to one or more parameters, as previously described, within permissible constraints, and may prevent the user from making inappropriate adjustments harmful to the user or the system 100. In one example, with reference to FIG. 2A, the display 120 may show a graphical user interface 160, illustrating a target pressure waveform 130 based on which the pressurized flow of breathable gas is generated while in the configuration mode. Thus, the user may adjust one or more parameters for controlling pressure therapy by adjusting one or more visual features on the target pressure waveform 130 and perceive the therapy (e.g., before and after the change) thus providing the user with a real time or near real time understanding of the change.

Thus, in the mode, the system 100 may simulate therapy based on the user's adjustment in real time or near real time as the user adjusts the parameter(s). For instance, whenever the user adjusts a parameter, the processor(s) 110 may detect the user's adjustment, and generate a sensory response (user feedback) perceivable by the user. In one example, the processor(s) 110 may detect the user's adjustment during a first respiratory cycle of the user while therapy is provided, and generate a sensory response based on the detected adjustment. The sensory response may include generating therapy, according to the adjustment, during one or more additional respiratory cycles of the user following the first respiratory cycle.

Thus, a sensory response may include the controller changing operation of the pressure generator 112 to adjust the pressurized flow of breathable gas based on the user's adjustment and delivering the adjusted pressurized flow of breathable gas to the patient interface (e.g., mask) worn by the user. Thus, the processor(s) 110 may detect the user's adjustment during a first respiratory cycle of the user, and adjust and deliver the pressurized flow of breathable gas to the user based on the detected adjustment during at least one, or more, respiratory cycle(s) of the user following the first respiratory cycle while in the configuration mode. As a result, the user may immediately feel (e.g., via the patient interface or mask) the effect of changes in the therapy as the user changes one or more parameters for controlling the therapy.

Additionally, or as an alternative, with continued reference to FIG. 2A, the processor(s) 110 may generate a visual response via the graphical user interface 160. The visual response may provide a real time view of one or more propagating or running waveforms resulted from the user's parameter adjustment. The visual response may display a first running waveform 162 corresponding to the adjusted pressurized flow of breathable gas generated by the pressure generator 112. The waveform 162 may begin at the beginning of inspiration ends at the end of expiration. The waveform 162 may, for example, be presented as a white curve on a black screen in the graphical user interface 160. The user may perceive by viewing, through the graphical user interface 160, how the waveform 162 changes in real time or near real time as the user adjusts relevant parameter(s).

In one example, the graphical user interface 160 may display a second running waveform 164 corresponding to the user's respiratory airflow. The user's respiratory airflow as indicated by the second running waveform 164 may represent what the user currently breathes in and out, which may change with each breath. The user's respiratory airflow may be detected by one or more sensors 124. The second running waveform 138 may be presented in a dashed curve, while the first running waveform 162 may be presented in a solid curve. The second running waveform may be displayed in an overlaying fashion with respect to the first running waveform, so that the user may visualize the user's actual breath relative to a simulated therapy waveform (when no therapy is provided) or a visual version of the actual therapy that is being provided by the system 100.

FIG. 2B provides another illustration of the graphical user interface 160 showing running waveforms 162 and 164 that encompass several respiratory cycles, which may advance across a display screen as they are produced over time.

FIG. 2C is another schematic illustration of the graphical user interface 160 showing a transition in shape of the pressure waveform 162, where the pressure waveform 162 transforms from a curved shape to a square-like shape as a result of an adjustment by the user in the configuration mode. The graphical user interface 160 also illustrates a second waveform 164 that represents the user's respiratory airflow relative to (i.e., on a common time scale as) the pressure waveform.

### 3. Further Technical Features and Advantages

### 3.1 Distributed Communications, Computing, and Data Synchronization

### 3.1.1 Wireless Device

Referring to FIG. 1D, the system 100 may be wirelessly connected with a wireless device 170, such as to achieve any operations described herein with regard to waveform personalization described in Section 1.2 and/or the operation of the set-up configuration mode when the wireless device 170 and therapy apparatus of the system 100 communicate with each other, such as by implementing any of the aforementioned user interface functionalities with the wireless device 170. As such, the wireless device 170 may be a computing system accessible by a user and may use a graphic user interface on the wireless device to activate the aforementioned processes and/or selections. Examples of the wireless device 170 may include mobile phone, tablet, netbook, desktop computer, laptop computer, and wearable computing device such as a smartwatch, among other possibilities. Referring to FIG. 1B, the wireless device 170 may include one or more processors 172, memory 174, a user interface 176 including a display 178, and a network interface 180. The network interface 180 may have one or more wireless transceivers, such as a Bluetooth transceiver, a cellular transceiver, and a Wi-Fi transceiver. The display 178 may be a monitor having a screen or any other electrical device that is operable to display information (e.g., text, imagery and/or other graphical elements). In addition, the wireless device 170 may include all of the components normally used in connection with a computing device such as a user interface subsystem. The user interface 176 may include one or more user input devices (e.g., a mouse, keyboard, touch screen and/or microphone) for receiving input from the user, and output devices such as speaker(s). The wireless device 170 may communicate, such as with the system 100, via any of the following transceivers: a Bluetooth transceiver, a cellular transceiver and a Wi-Fi transceiver.

When the wireless device 170 is communicatively connected with the system 100, the wireless device 170 may have two-way communication with the system 100. The wireless device 170 may transmit any user input, including any selection of a waveform, response to a prompt, and/or adjustment to one or more waveform parameters, to the system 100. The system 100 may receive the user's input via the wireless device 170. The pressure generator 112 of the system 100 may adjust the therapy based on the user's input to device 170. The system 100 may send information related to the adjusted therapy, the user's respiratory airflow, user prompts, and any other information that would otherwise be available via user interface 116 to the wireless device 170 and may request the wireless device 170 to display any visual or other response to the user. The wireless device 170 may generate a visual or audio response to the user based on the received information.

Wireless device 170 may be used in any of the waveform personalization aspects described above in Section 1.2 and elsewhere herein. Moreover, wireless device 170 may be configured to perform any of the features of processor(s) 110 described herein.

Although device 170 is described as being a wireless device, it is to be appreciated that in other aspects, device 170 may be a device (e.g., such as a laptop or desktop computer) that is configured to communicate with system 100 via a wired connection and using any suitable wired communication protocol.

### 3.1.2 Server(S) and Cloud Connectivity

As shown in FIG. 1B, system 100 and/or wireless device 170 may further be communicatively connected (e.g., via the Internet, a cellular network, or any other network) to one or more server(s) 190. The server(s) 190 may include one or more processor(s) 192, one or more memory 194, user interface 196 (including display 198), and network interface 199. Processor(s) 192 may include and be configured to perform any of the features of processor(s) 110, 172 described herein. Memory 194 may include any of the features and store any of the data described herein in relation to memory 114, 174. User interface 196 may include any of the features of user interfaces 116, 176. Network interface 199 may include any of the features of network interfaces 118, 180.

In some aspects, server(s) 190, system 100, and wireless device 170 are configured to communicate to exchange data. In one aspect, server(s) 190 are configured to receive (via network interface 199) data from system 100 and/or wireless device 170. The data may include any of the data (i) entered into user interfaces 116, 176, (ii) generated by processor(s) 110, 172, (iii) stored in memory 114, 174, and/or (iii) outputted by sensor(s) 124. The data may be stored in memory 194 and processor(s) 192 may process the data to generate processed data. The processed data generated by processor(s) 192 is sent to system 100 and/or wireless device 170. In some aspects, processor(s) 192 may be configured to process the data received from system 100 and/or wireless device 170 to replace some or all of the functions of processor(s) 110 and/or processor(s) 172. For example, processor(s) 192 of server(s) 190 may receive the data inputted into user interfaces 116 and/or 176 and the data from sensor(s) 124 and may perform any of the personalized waveform processes described herein, such as determining the waveform parameters for a personalized waveform or an adjustment to a target waveform to personalize the target waveform. Processor(s) 192 may be configured to transmit the generated or adjusted personalized waveforms (i.e., the waveform parameters associated with each) to wireless device 170 and/or system 100, so that system 100 may then use the personalized waveforms to deliver a pressurized flow of breathable gas to the user according to the personalized waveforms determined or identified by processor(s) 192. It is to be appreciated that server(s) 190 may communicate with, and process data for, a plurality of systems 100 (and/or wireless devices 170). An advantage of using server(s) 190 to process data is that the computational and processing load in relation to the waveform personalization techniques described herein may be offloaded to the processor(s) of the server(s) 190 rather than being placed on the user devices (e.g., system 100 and wireless device 170), which may have comparatively less computational capabilities than the server(s) 190.

In one aspect, the user's personalized waveforms (and any other associated data) may be stored in and shared between system memory 114, wireless device memory 174, and/or server memory 194. In this regard, when the user uses any of the waveform personalization aspects described herein to identify personalized waveforms, the personalized waveforms may be stored in any one of system memory 114, wireless device memory 174, and/or server memory 194. Thereafter, system 100, wireless device 170, and/or server(s) 190 are configured to share and synchronize the data (e.g., waveform parameter values) associated with each of the personalized waveforms stored in each of system 100, wireless device 170, and/or server(s) 190. The personalized waveform data (and any other data associated with the user, such as user credentials, device settings and preferences, etc.) may be associated to a user profile. In this way, if a user replaces their system 100, the user's personalized waveform data (and any other data associated with the user) can be transferred from the memory 194 of server(s) 190 or the memory 174 of wireless device 170 when the user logs into their user profile (e.g., on their wireless device 170 and/or on system 100) so it may then be used on a new system 100. The data transfer process may occur automatically, e.g., when system 100 is first setup and attempts to synchronize the user data, settings, and/or preferences it stores in memory 114 with the user data, settings, and/or preferences of the same user stored in memory 174 and/or memory 194. Alternatively, the data transfer process may occur in response to a request from the user (e.g., via input to user interface 176 or user interface 116).

### 3.2 System Control

It is to be appreciated that system 100 may be configured to be controlled in many different ways. For example, as described above, system 100 may be controlled and interacted with via user input (e.g., using buttons, touch screens, etc.) to any one of user interfaces 116, 176, 196. In some aspects, system 100, wireless device 170, and/or server(s) 190 may be configured to receive audio (i.e., spoken) commands or input from a user as a means of interacting with and controlling system 100. In one example, system 100 and/or wireless device 170 may include a microphone and processor(s) 110, 172 may be configured to convert the audio information received by the microphone into commands or user input. System 100 and/or wireless device 170 may also include a speaker for responding via audio output by the speaker to received voice commands and inputs from the user or for outputting audio prompts, queries, or notifications. In this manner, audio and audio devices (e.g., microphone and/or speaker) may be used as a medium that can be part of a user interface for interaction between (i) system 100 and/or wireless device 170 and (ii) the user. In this aspect, processor(s) 110, 172 are configured to perform any of the waveform personalization aspects described herein by communicating with the user by audio or voice commands and responses (i.e., receiving audio commands from the user into the microphone and outputting audio responses, queries, prompts, etc. using the speaker). For example, voice commands may be received by the system 100 or wireless device 170 to initiate or select between any one of the waveform personalization processes described herein. Moreover, system 100 or wireless device 170 may prompt for and wait to hear for input from the user regarding whether they find a personalized waveform comfortable or what aspects of the waveform they found comfortable or uncomfortable. The audio user input may then be used to perform the waveform personalization processes described above (such as performing A/B waveform testing).

### 3.3 Technical Advantages

The disclosed technology may have many technical advantages. First, the disclosed technology may place therapy control in the user's hands. The disclosed technology may enable the user to adjust therapy with complete ease and confidence. By using the disclosed technology, the user may independently find the ideal therapy parameter setting(s) tailored to the user's needs and/or comfortable level, without reliance on any clinical support.

Second, the disclosed technology may provide sensory response(s) in real time or near real time to the user as the user adjusts one or more parameters related to therapy. For example, the user can feel in the user's respiratory system a tangible difference for each parameter adjustment. As a result, the user can easily decide what parameter setting(s) makes the user feel most comfortable. The disclosed technology may also provide a visual response to the user via a display showing effects of the user's adjustment.

Third, by enabling adjustments of visual features associated with therapy via a touch screen, the disclosed technology provides greater degree of freedom for adjusting parameters, and allows the user to adjust parameters in without a high degree of technical understanding.

Fourth, automated adjustment and control of the waveform parameters described above enable a high degree of personalization of the waveform provided to the user in a fast (real time or near real time) manner such that a waveform may be personalized to the user's unique breathing pattern and preferences.

Fifth, the usage of sensor based feedback loops, as is utilized in Sections 1.2.4.1 and 1.2.4.2 may overcome issues that may arise with user's that are not able to identify or characterize what particular aspects of their breathing or a waveform is uncomfortable.

### 3.4 Example Memories and Processors

The memory 114, 174 and 194 may be databases that store information accessible by the processor(s) 110, 172 and 192, respectively. For example, the memory 114, 174 and 194 may store instructions and data associated with adjustable waveform and other parameters for controlling pressure support generated by the pressure generator 112, such as to generate user interface(s) for such waveform customizations described herein. The memory 174 of the wireless device 170 and memory 194 of server(s) 190 may store instructions and data received from the system 100. The memory 114, 174 and 194 may be of any type capable of storing information accessible by the processor(s), including a computing device-readable medium. The memory may be a non-transitory medium such as a hard-drive, memory card, optical disk, solid-state, etc. The memory may include different combinations of the foregoing, whereby different portions of the instructions and data are stored on different types of media. The instructions may be any set of instructions to be executed directly (such as machine code) or indirectly (such as scripts) by the processor(s). For example, the instructions may be stored as computing device code on the computing device-readable medium. In that regard, the terms “instructions”, “modules” and “programs” may be used interchangeably herein. The instructions may be stored in object code format for direct processing by the processor, or in any other computing device language including scripts or collections of independent source code modules that are interpreted on demand or compiled in advance.

The processor(s) 110, 172, and 192 may be any conventional processors, such as commercially available GPUs, CPUs, TPUs, etc. Alternatively, each processor may be a dedicated device such as an ASIC or other hardware-based processor. Although FIG. 1B functionally illustrates the processors, memory as being within the same block, such devices may actually include multiple processors, computing devices, or memories that may or may not be stored within the same physical housing. Similarly, the memory may be a hard drive or other storage media located in a housing different from that of the processor(s), for instance in a cloud computing system. Accordingly, references to a processor or computing device will be understood to include references to a collection of processors or computing devices or memories that may or may not operate in parallel. The processor(s) 110, 172, and 192 may respectively access the memory 114, 174, and 194 via a network.

### 4.1 Example Treatment Systems

An example embodiment of the system 100 is discussed in more detail in the following sections 4.1 to 4.5.

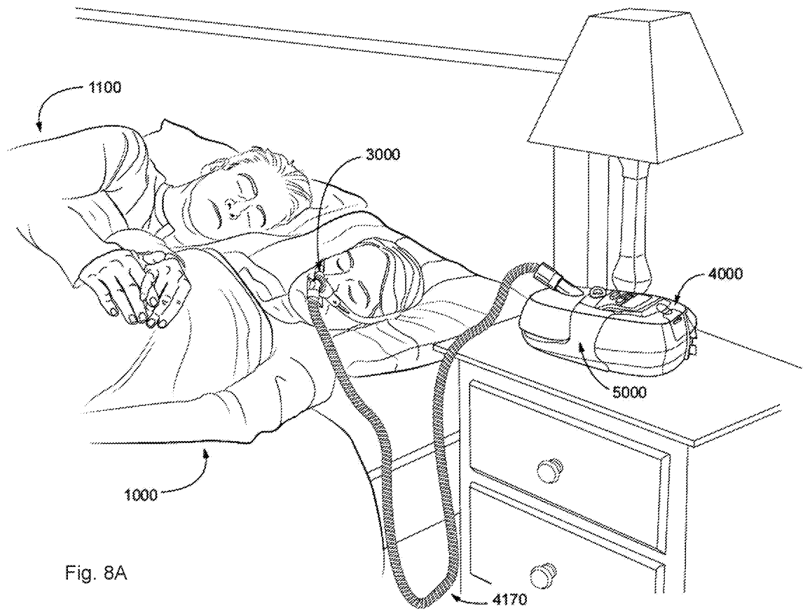

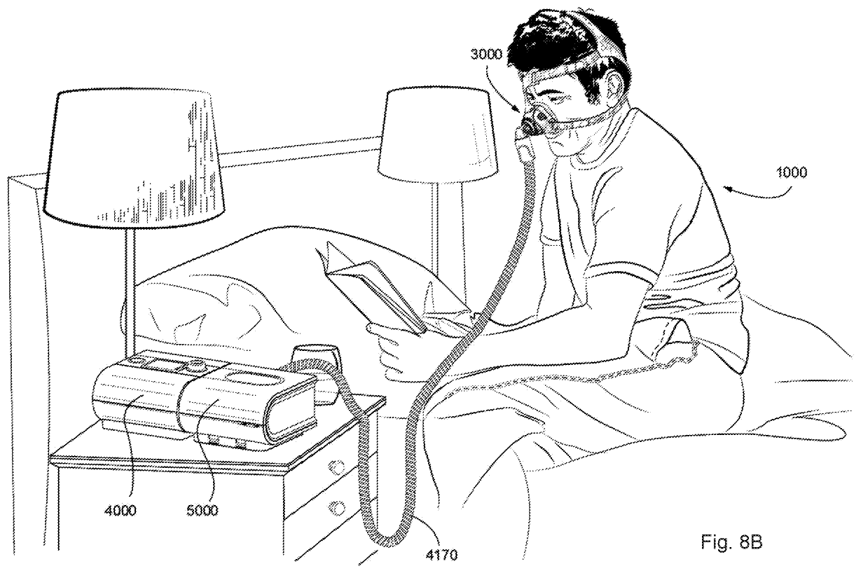

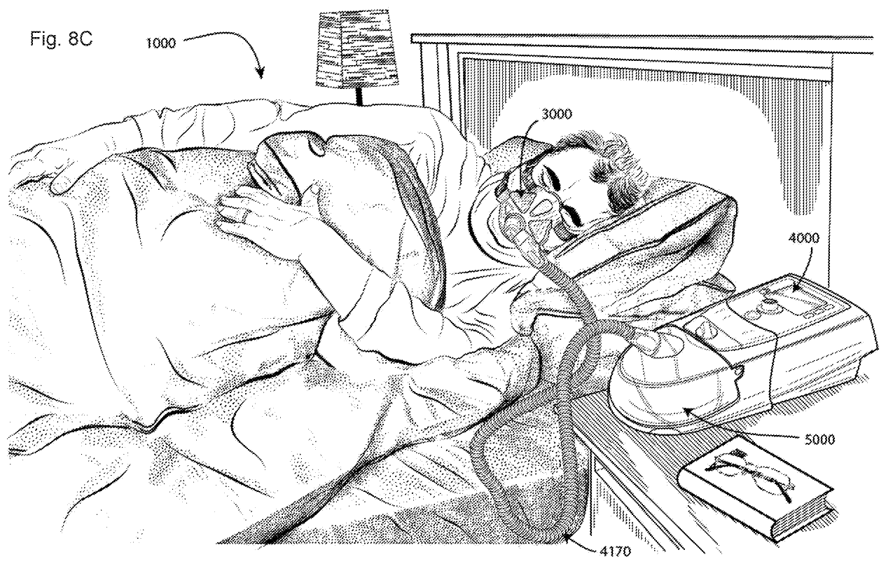

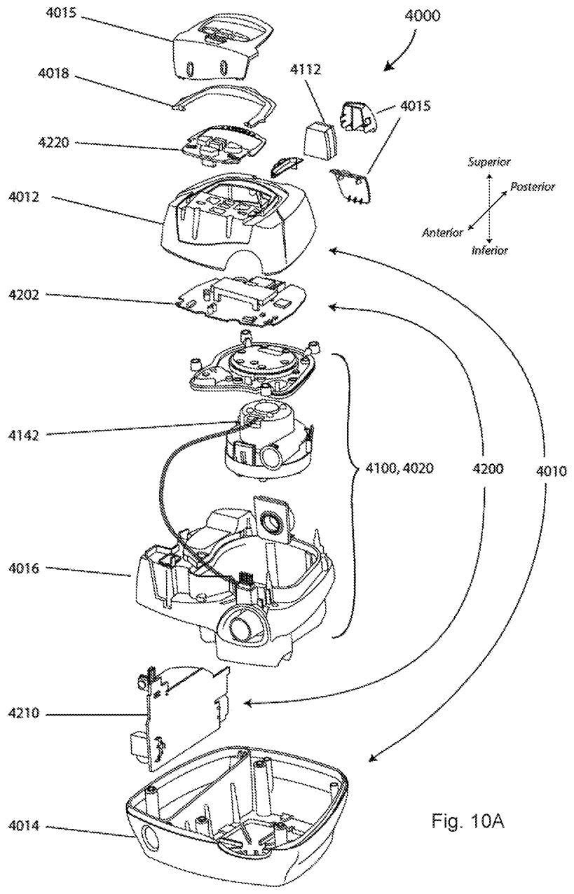

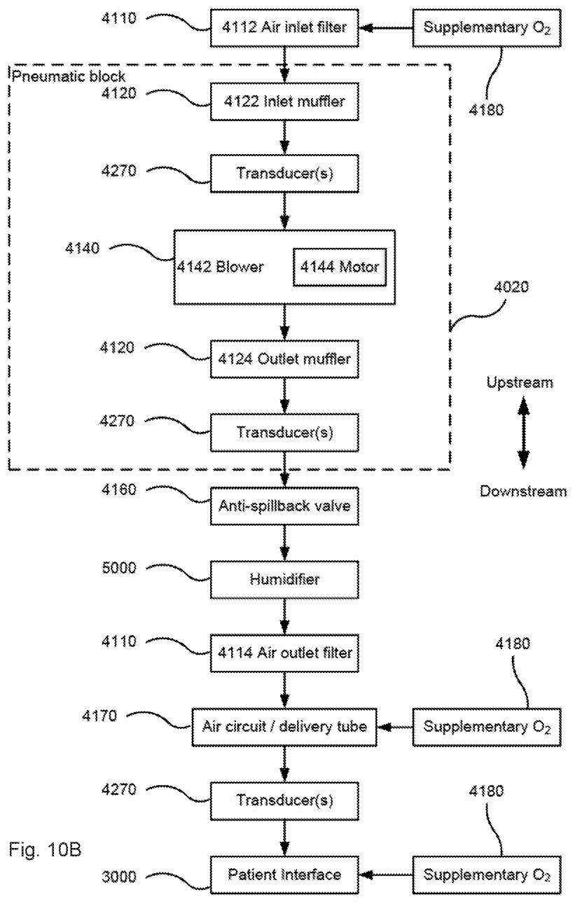

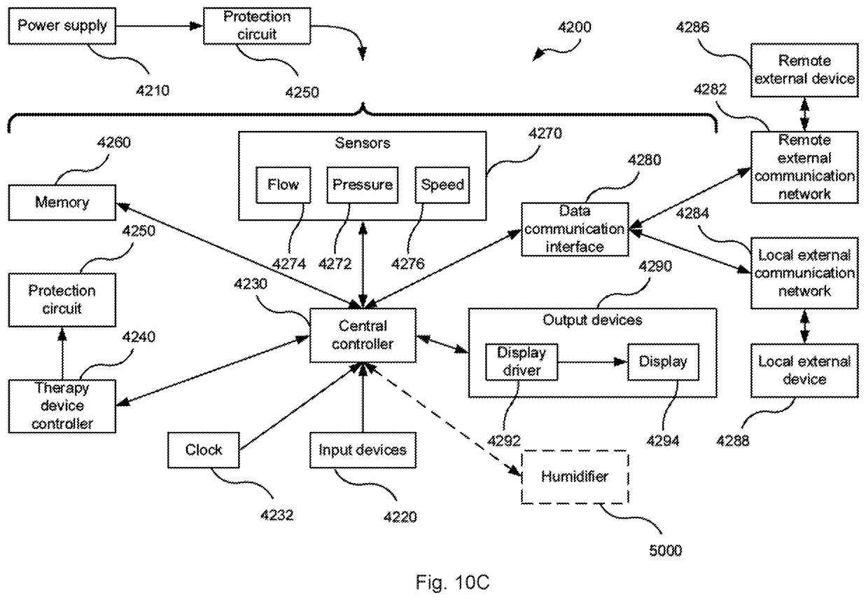

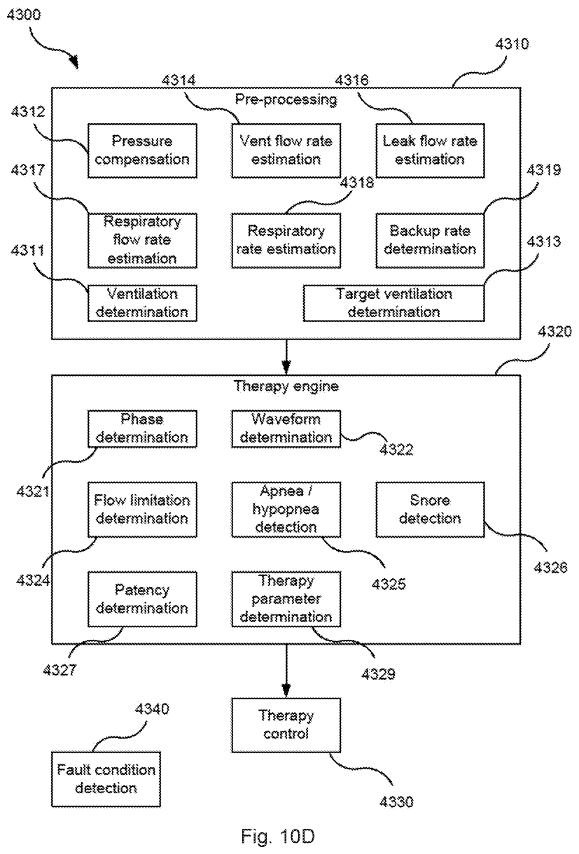

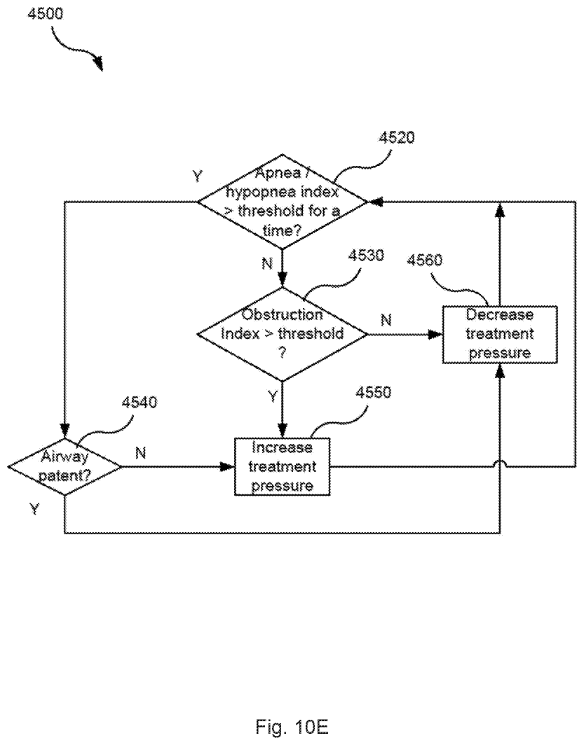

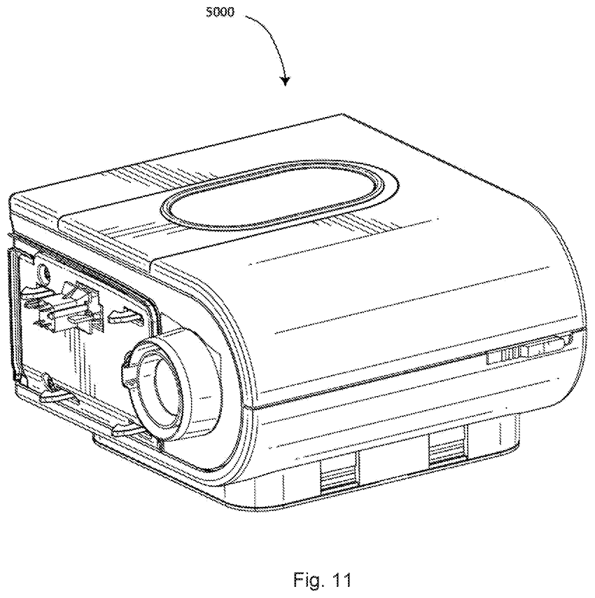

In one form, the system 100 may treat and/or monitor a respiratory disorder. The system 100 may be a respiratory therapy device (RT) such as an RPT device 4000 for supplying a flow of pressurised air to the patient 1000 via an air circuit 4170 leading to a patient interface 3000. The flow of air may be pressure-controlled (for respiratory pressure therapies) or flow-controlled (for flow therapies such as high flow therapy HFT). Thus, RPT devices may also be configured to act as flow therapy devices, such as when using a patient interface that does not use a seal that seals with the patient's respiratory system. In the following description, the RT or RPT device may be considered in reference to FIGS. 8A-11.

### 4.2 Patient Interface

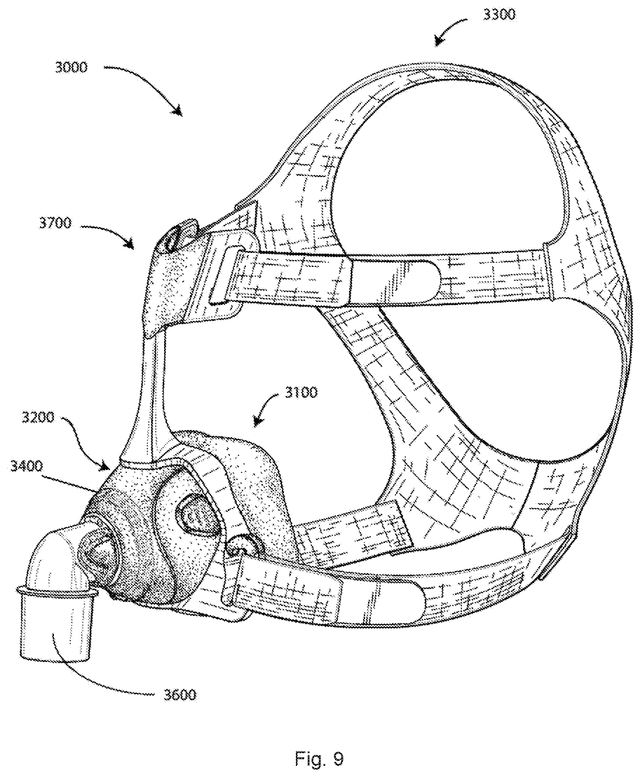

As shown in FIG. 9, a non-invasive patient interface 3000 in accordance with one aspect of the present technology may optionally comprise any of the following functional aspects: a seal-forming structure 3100, a plenum chamber 3200, a positioning and stabilising structure 3300, a vent 3400, a connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to an airway of the patient so as to facilitate the supply of pressurised air to the airway.

### 4.3 RPT Device

An RPT device 4000 in accordance with one aspect of the present technology comprises mechanical and pneumatic components 4100, electrical components 4200 and is programmed to execute one or more algorithms 4300. The RPT device 4000 may have an external housing 4010 formed in two parts, an upper portion 4012 and a lower portion 4014. In one form, the external housing 4010 may include one or more panel(s) 4015. The RPT device 4000 may comprise a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying pressurised air (e.g., a blower 4142), an outlet muffler 4124, and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 4.3.1 RPT Device Mechanical & Pneumatic Components

An RPT device 4000 may comprise one or more of the following components in an integral unit. In an alternative form, one or more of the following components may be located as respective separate units.

### 4.3.1.1 Air Filter(s)

An RPT device 4000 In Accordance with One Form of the Present Technology May include an air filter 4110, or a plurality of air filters 4110.

In one form, an air inlet filter 4112 is located at the beginning of the pneumatic path upstream of a pressure generator 4140.

In one form, an air outlet filter 4114, for example an antibacterial filter, is located between an outlet of the pneumatic block 4020 and a patient interface 3000.

### 4.3.1.2 Muffler(s)

An RPT device 4000 in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000.

### 4.3.1.3 Pressure Generator

In one form of the present technology, a pressure generator 4140 for supplying pressurised air is a controllable blower 4142. For example, the blower 4142 may include a brushless DC motor 4144 with one or more impellers housed in a volute. The pressure generator 4140 may be capable of generating a supply or flow of air, for example at about 120 litres/minute, at a positive pressure in a range from about 4 cmH₂O to about 20 cmH₂O, or in other forms up to about 30 cmH₂O.

The pressure generator 4140 is under the control of the therapy device controller 4240.

In other forms, a pressure generator 4140 may be a piston-driven pump, a pressure regulator connected to a high pressure source (e.g., compressed air reservoir), or a bellows.

### 4.3.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 are constructed and arranged to generate data representing respective properties of the air flow, such as a flow rate, a pressure or a temperature, at that point in the pneumatic path.

In one form of the present technology, one or more transducers 4270 are located proximate to the patient interface 3000.

In one form, a signal from a transducer 4270 may be filtered, such as by low-pass, high-pass or band-pass filtering.

### 4.3.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 4.3.1.6 Air Circuit

An air circuit 4170 in accordance with one aspect of the present technology is a conduit or tube constructed and arranged to allow, in use, a flow of air to travel between two components such as the pneumatic block 4020 and the patient interface 3000.

### 4.3.1.7 Oxygen Delivery

In one form of the present technology, supplemental oxygen 4180 is delivered to one or more points in the pneumatic path, such as upstream of the pneumatic block 4020, to the air circuit 4170 and/or to the patient interface 3000.

### 4.3.2 RPT Device Electrical Components

### 4.3.2.1 Power Supply

In one form of the present technology power supply 4210 is internal of the external housing 4010 of the RPT device 4000. In another form of the present technology, power supply 4210 is external of the external housing 4010 of the RPT device 4000.

In one form of the present technology power supply 4210 provides electrical power to the RPT device 4000 only. In another form of the present technology, power supply 4210 provides electrical power to both RPT device 4000 and humidifier 5000.

### 4.3.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form the input device 4220 may be constructed and arranged to allow a person to select a value and/or a menu option.

### 4.3.2.3 Central Controller

In one form of the present technology, the central controller 4230 is a Processor suitable to control an RPT device 4000 such as an x86 INTEL processor.

A central controller 4230 suitable to control an RPT device 4000 in accordance with another form of the present technology includes a processor based on ARM Cortex-M processor from ARM Holdings. For example, an STM32 series microcontroller from ST MICROELECTRONICS may be used.

Another central controller 4230 suitable to control an RPT device 4000 in accordance with a further alternative form of the present technology includes a member selected from the family ARM9-based 32-bit RISC CPUs. For example, an STR9 series microcontroller from ST MICROELECTRONICS may be used.

In certain alternative forms of the present technology, a 16-bit RISC CPU may be used as the central controller 4230 for the RPT device 4000. For example, a processor from the MSP430 family of microcontrollers, manufactured by TEXAS INSTRUMENTS, may be used.

In another form of the present technology, the central controller 4230 is a dedicated electronic circuit. In another form, the central controller 4230 is an application-specific integrated circuit (ASIC). In another form, the central controller 4230 comprises discrete electronic components.

The central controller 4230 is configured to receive input signal(s) from one or more transducers 4270, one or more input devices 4220, and the humidifier 5000.

The central controller 4230 is configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms 4300, expressed as computer programs stored in a non-transitory computer readable storage medium, such as memory 4260 or other memory described herein. In some forms of the present technology, as previously discussed, the central controller 4230 may be integrated with an RPT device 4000. However, in some forms of the present technology, some methodologies may be performed by a remotely located device or server such as the server previously mentioned. For example, the remotely located device or server may determine control settings for transfer to a ventilator or other RT device such as by detecting respiratory related events and distinguishing them by type by an analysis of stored data such as from any of the sensors described herein.

While the central controller 4230 may comprise a single controller interacting with various sensors 4270, data communications interface 4280, memory 4260, as well as other devices, the functions of controller 4230 may be distributed among more than one controller. Thus, the term “central” as used herein is not meant to limit the architecture to a single controller or processor that controls the other devices. For example, alternative architectures may include a distributed controller architecture involving more than one controller or processor, which may optionally be directly or indirectly in electronic (wired or wireless) communications with the previously described finger sensor or a server in communication with the finger sensor, such as for implementing any of the methodologies described herein. This may include, for example, a separate local (i.e., within RPT device 4000) or remotely located controller that perform some of the algorithms 4300, or even more than one local or remote memory that stores some of the algorithms. In addition, the algorithms when expressed as computer programs may comprise high level human readable code (e.g., C++, Visual Basic, other object oriented languages, etc.) or low/machine level instructions (Assembler, Verilog, etc.). Depending on the functionality of an algorithm(s), such code or instructions may be burnt in the controller, e.g., an ASIC or DSP, or be a run time executable ported to a DSP or general purpose processor that then becomes specifically programmed to perform the tasks required by the algorithm(s).

### 4.3.2.4 Clock

The RPT device 4000 may include a clock 4232 that is connected to the central controller 4230.

### 4.3.2.5 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 4.3.2.6 Protection Circuits

An RPT device 4000 in accordance with the present technology may comprise one or more protection circuits 4250.

One form of protection circuit 4250 in accordance with the present technology is an electrical protection circuit.

One form of protection circuit 4250 in accordance with the present technology is a temperature or pressure safety circuit.

### 4.3.2.7 Memory

In accordance with one form of the present technology the RPT device 4000 includes memory 4260, for example non-volatile memory. In some forms, memory 4260 may include battery powered static RAM. In some forms, memory 4260 may include volatile RAM.

Memory 4260 may be located on PCBA 4202. Memory 4260 may be in the form of EEPROM, or NAND flash.

Additionally or alternatively, RPT device 4000 includes a removable form of memory 4260, for example a memory card made in accordance with the Secure Digital (SD) standard.

In one form of the present technology, the memory 4260, such as any of the memories previously described, acts as a non-transitory computer readable storage medium on which is stored computer program instructions expressing the one or more methodologies described herein, such as the one or more algorithms 4300.

### 4.3.2.8 Transducers

Transducers may be internal of the device 4000, or external of the RPT device 4000. External transducers may be located for example on or form part of the air delivery circuit 4170, e.g., at the patient interface 3000. External transducers may be in the form of non-contact sensors such as a Doppler radar movement sensor that transmit or transfer data to the RPT device 4000.

### 4.3.2.8.1 Flow Rate

A flow rate transducer 4274 in accordance with the present technology may be based on a differential pressure transducer, for example, an SDP600 Series differential pressure transducer from SENSIRION. The differential pressure transducer is in fluid communication with the pneumatic circuit, with one of each of the pressure transducers connected to respective first and second points in a flow restricting element.

In one example, a signal representing total flow rate Qt from the flow transducer 4274 is received by the central controller 4230.

### 4.3.2.8.2 Pressure

A pressure transducer 4272 in accordance with the present technology is located in fluid communication with the pneumatic path. An example of a suitable pressure transducer 4272 is a sensor from the HONEYWELL ASDX series. An alternative suitable pressure transducer is a sensor from the NPA Series from GENERAL ELECTRIC.

In use, a signal from the pressure transducer 4272 is received by the central controller 4230. In one form, the signal from the pressure transducer 4272 is filtered prior to being received by the central controller 4230.

### 4.3.2.8.3 Motor Speed

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 4.3.2.9 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g., via Ethernet, or optical fibre) or a wireless protocol (e.g., CDMA, GSM, LTE) to connect to the Internet.

In one form, local external communication network 4284 utilises one or more communication standards, such as Bluetooth, or a consumer infrared protocol and may optionally communicate with any of the sensors described herein.

In one form, remote external device 4286 is one or more computers, for example a cluster of networked computers and/or server as described herein. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 4.3.2.10 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 4.3.2.10.1 Display Driver

A display driver 4292 receives as an input the characters, symbols, or images intended for display on the display 4294, and converts them to commands that cause the display 4294 to display those characters, symbols, or images.

### 4.3.2.10.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 4.3.3 RPT Device Algorithms

### 4.3.3.1 Pre-Processing Module

A pre-processing module 4310 in accordance with the present technology receives, as an input, raw data from a transducer 4270, for example a flow rate sensor 4274 or a pressure sensor 4272, and performs one or more process steps to calculate one or more output values that will be used as an input to another module, for example a therapy engine module 4320.

In one form of the present technology, the output values include the interface or mask pressure Pm, the respiratory flow rate Qr, and the leak flow rate Ql.

In various forms of the present technology, the pre-processing module 4310 comprises one or more of the following algorithms: pressure compensation 4312, vent flow rate estimation 4314, leak flow rate estimation 4316, respiratory flow rate estimation 4317, ventilation determination 4311, target ventilation determination 4313, respiratory rate estimation 4318, and backup rate determination 4319.

### 4.3.3.1.1 Pressure Compensation

In one form of the present technology, a pressure compensation algorithm 4312 receives as an input a signal indicative of the pressure in the pneumatic path proximal to an outlet of the pneumatic block 4020. The pressure compensation algorithm 4312 estimates the pressure drop in the air circuit 4170 and provides as an output an estimated pressure, Pm, in the patient interface 3000.

### 4.3.3.1.2 Vent Flow Rate Estimation

In one form of the present technology, a vent flow rate estimation algorithm 4314 receives as an input an estimated pressure, Pm, in the patient interface 3000 and estimates a vent flow rate of air, Qv, from a vent 3400 in a patient interface 3000.

### 4.3.3.1.3 Leak Flow Rate Estimation

In one form of the present technology, a leak flow rate estimation algorithm 4316 receives as an input a total flow rate Qt and a vent flow rate Qv, and estimates a leak flow rate Ql. In one form, the leak flow rate estimation algorithm 4316 estimates the leak flow rate Ql by calculating an average of the difference between the total flow rate and the vent flow rate Qv over a period sufficiently long to include several breathing cycles, e.g., about 10 seconds.

In one form, the leak flow estimation algorithm 4316 receives as an input a total flow rate Qt, a vent flow rate Qv, and an estimated pressure, Pm, in the patient interface 3000, and estimates a leak flow rate Ql by calculating a leak conductance, and determining a leak flow rate Ql to be a function of leak conductance and the pressure Pm. Leak conductance may be calculated as the quotient of low-pass filtered non-vent flow rate equal to the difference between total flow rate Qt and vent flow rate Qv, and low-pass filtered square root of pressure Pm, where the low-pass filter time constant has a value sufficiently long to include several breathing cycles, e.g., about 10 seconds. The leak flow rate Ql may be estimated as the product of leak conductance and a function of pressure, Pm.

### 4.3.3.1.4 Respiratory Flow Rate Estimation

In one form of the present technology, a respiratory flow rate estimation algorithm 4317 receives as an input a total flow rate, Qt, a vent flow rate, Qv, and a leak flow rate, Ql, and estimates a respiratory flow rate of air, Qr, to the patient, by subtracting the vent flow rate Qv and the leak flow rate Ql from the total flow rate Qt.

In other forms of the present technology, the respiratory flow estimation algorithm 4317 provides a value that acts as a proxy for the respiratory flow rate Qr. Possible proxies for respiratory flow rate include:
Respiratory movement of the chest of the patient 1000
Current drawn by the pressure generator 4140
Motor speed of the pressure generator 4140
Trans-thoracic impedance of the patient 1000

The respiratory flow rate proxy value may be provided by a transducer 4270 in the RPT device 4000, e.g., the motor speed sensor 4276, or a sensor external to the RPT device 4000, such a respiratory movement sensor or a trans-thoracic impedance sensor.

### 4.3.3.1.5 Ventilation Determination

In one form of the present technology, a ventilation determination algorithm 4311 receives an input a respiratory flow rate Qr, and determines a measure Vent indicative of current patient ventilation.

In some implementations, the ventilation determination algorithm 4311 determines a measure of ventilation Vent that is an estimate of actual patient ventilation.

In one such implementation, the measure of ventilation Vent is half the absolute value of respiratory flow, Qr, optionally filtered by low-pass filter such as a second order Bessel low-pass filter with a corner frequency of 0.11 Hz.

In one such implementation, the measure of ventilation Vent is an estimate of gross alveolar ventilation (i.e. non-anatomical-deadspace ventilation). This requires an estimate of anatomical deadspace. One can use the patient's height (or arm-span in cases of severe skeletal deformity) as a good predictor of anatomical deadspace. Gross alveolar ventilation is then equal to a measure of actual patient ventilation, e.g., determined as above, less the product of the estimated anatomical deadspace and the estimated spontaneous respiratory rate Rs.

In other implementations, the ventilation determination algorithm 4311 determines a measure of ventilation Vent that is broadly proportional to actual patient ventilation. One such implementation estimates peak respiratory flow rate Qpeak over the inspiratory portion of the cycle. This and many other procedures involving sampling the respiratory flow rate Qr produce measures which are broadly proportional to ventilation, provided the flow rate waveform shape does not vary very much (here, the shape of two breaths is taken to be similar when the flow rate waveforms of the breaths normalised in time and amplitude are similar). Some simple examples include the median positive respiratory flow rate, the median of the absolute value of respiratory flow rate, and the standard deviation of flow rate. Arbitrary linear combinations of arbitrary order statistics of the absolute value of respiratory flow rate using positive coefficients, and even some using both positive and negative coefficients, are approximately proportional to ventilation. Another example is the mean of the respiratory flow rate in the middle K proportion (by time) of the inspiratory portion, where 0<K<1. There is an arbitrarily large number of measures that are exactly proportional to ventilation if the flow rate waveform shape is constant.

In other forms, the ventilation determination algorithm 4311 determines a measure Vent of ventilation that is not based on respiratory flow rate Qr, but is a proxy for the current patient ventilation, such as oxygen saturation (SaO₂), or partial pressure of carbon dioxide (PCO₂), obtained from suitable sensors attached to the patient 1000.

### 4.3.3.1.6 Target Ventilation Determination

In one form of the present technology, a central controller 4230 takes as input the measure of current ventilation, Vent, and executes one or more target ventilation determination algorithms 4313 for the determination of a target value Vtgt for the measure of ventilation.

In some forms of the present technology, there is no target ventilation determination algorithm 4313, and the target ventilation Vtgt is predetermined, for example by hard-coding during configuration of the RPT device 4000 or by manual entry through the input device 4220.

In other forms of the present technology, such as adaptive servo-ventilation (ASV) therapy (described below), the target ventilation determination algorithm 4313 computes the target ventilation Vtgt from a value Vtyp indicative of the typical recent ventilation of the patient 1000.

In some forms of adaptive servo-ventilation therapy, the target ventilation Vtgt is computed as a high proportion of, but less than, the typical recent ventilation Vtyp. The high proportion in such forms may be in the range (80%, 100%), or (85%, 95%), or (87%, 92%).

In other forms of adaptive servo-ventilation therapy, the target ventilation Vtgt is computed as a slightly greater than unity multiple of the typical recent ventilation Vtyp.

The typical recent ventilation Vtyp is the value around which the distribution of the measure of current ventilation Vent over multiple time instants over some predetermined timescale tends to cluster, that is, a measure of the central tendency of the measure of current ventilation over recent history. In one implementation of the target ventilation determination algorithm 4313, the recent history is of the order of several minutes, but in any case should be longer than the timescale of Cheyne-Stokes waxing and waning cycles. The target ventilation determination algorithm 4313 may use any of the variety of well-known measures of central tendency to determine the typical recent ventilation Vtyp from the measure of current ventilation, Vent. One such measure is the output of a low-pass filter on the measure of current ventilation Vent, with time constant equal to one hundred seconds.

### 4.3.3.1.7 Respiratory Rate Estimation

In one form of the present technology, a respiratory rate estimation algorithm 4318 receives as an input a respiratory flow rate, Qr, to the patient 1000, and produces an estimate of the spontaneous respiratory rate Rs of the patient.

The respiratory rate estimation algorithm 4318 may estimate the spontaneous respiratory rate Rs over periods when the patient 1000 is breathing spontaneously, i.e., when the RPT device 4000 is not delivering “backup breaths” (described below). In some forms of the present technology, the respiratory rate estimation algorithm 4318 estimates the respiratory rate over periods when servo-assistance (defined as pressure support minus minimum pressure support) is low, in one implementation less than 4 cmH₂O, as such periods are more likely to reflect spontaneous respiratory effort.

In some forms of the present technology, the respiratory rate estimation algorithm 4318 estimates the respiratory rate over periods of asleep breathing, since the respiratory rate during these periods may be substantially different from the respiratory rate during wake. Anxiety typically results in a higher respiratory rate than that prevailing during sleep. When patients focus on their own breathing process, their respiratory rates are typically lower than those during normal wakefulness or during sleep. Techniques such as described in Patent Application no. PCT/AU2010/000894, published as WO 2011/006199, the entire disclosure of which is hereby incorporated herein by reference, may be used to identify periods of awake breathing from the respiratory flow rate, Qr.

In some forms of the present technology, the respiratory rate estimation algorithm 4318 estimates the spontaneous respiratory rate Rs as the reciprocal of one of a variety of well-known statistical measures of central tendency of breath duration Ttot during the period of interest. In such measures it is desirable to reject, or at least be robust to, outliers. One such measure, trimmed mean, in which the lower and upper K proportions of the sorted breath durations are discarded and the mean calculated on the remaining breath durations, is robust to outliers. For example, when K is 0.25, this amounts to discarding the upper and lower quartiles of breath duration Ttot. The median is another robust measure of central tendency, though this can occasionally give unsatisfactory results when the distribution is strongly bimodal. A simple mean may also be employed as a measure of central tendency, though it is sensitive to outliers. An initial interval filtering stage, in which contiguous time intervals corresponding to implausible respiratory rates (e.g., greater than 45 breaths/minute or less than 6 breaths/minute) are excluded as outliers from the mean calculation, may be employed. Other filtering mechanisms which may be used alone or in combination with interval filtering are to exclude any breaths that are not part of a sequence of N successive spontaneous breaths, where N is some small integer (e.g., 3), and to exclude the early and late breaths of a sequence of successive spontaneous breaths, e.g., to exclude the first and last breaths of a sequence of four breaths. The rationale for the latter mechanism is that the first and the last breaths in particular, and the early and late breaths in general, of a sequence of spontaneous breaths may be atypical; for example, the first spontaneous breath may occur as a result of an arousal, and the last spontaneous breath may be longer because of the decreasing respiratory drive which results in the backup breath which ends the sequence of spontaneous breaths.

In some forms of the present technology, the respiratory rate estimation algorithm 4318 makes an initial estimate of the spontaneous respiratory rate Rs using an initial period of estimation, to enable the subsequent processing in the therapy engine module 4320 to begin, and then continuously updates the estimate of the spontaneous respiratory rate Rs using a period of estimation that is longer than the initial period of estimation, to improve statistical robustness. For example, the initial period of estimation may be 20 minutes of suitable spontaneous breaths, but the period of estimation may then progressively increase up to some maximum duration, for example 8 hours. Rather than a rolling window of this duration being used for this estimation, low-pass filters on breath duration may be used, with progressively longer response times (more precisely, progressively lower corner frequencies) as the session proceeds.

In some forms, a suitably processed short-term (e.g., 10-minute) measure of central tendency, such as trimmed mean, may be input to a suitable low-pass filter to give an estimate Rs which changes on the time scale of hours or longer. This has the advantage that potentially large amounts of breath duration data do not need to be stored and processed, as might occur if a trimmed mean needs to be calculated on a moving window of breath duration data lasting hours or days.

In some forms of the present technology, respiratory rates measured over short periods of time, and in particular over one breath, may also be used instead of breath duration in the above-described measures of central tendency, giving generally similar but not identical results.

### 4.3.3.2 Therapy Engine Module

In one form of the present technology, a therapy engine module 4320 receives as inputs one or more of a pressure, Pm, in a patient interface 3000, a respiratory flow rate of air to a patient, Qr, and an estimate Rs of the spontaneous respiratory rate, and provides as an output one or more therapy parameters. In various forms, the therapy engine module 4320 comprises one or more of the following algorithms: phase determination 4321, waveform determination 4322, inspiratory flow limitation determination 4324, apnea/hypopnea determination 4325, snore detection 4326, airway patency determination 4327, and therapy parameter determination 4329.

### 4.3.3.2.1 Phase Determination

In one form of the present technology, a phase determination algorithm 4321 receives as an input a signal indicative of respiratory flow, Qr, and provides as an output a phase Φ of a current breathing cycle of a patient 1000.

In some forms, known as discrete phase determination, the phase output Φ is a discrete variable. One implementation of discrete phase determination provides a bi-valued phase output Φ with values of either inhalation or exhalation, for example represented as values of 0 and 0.5 revolutions respectively, upon detecting the start of spontaneous inhalation and exhalation respectively. RPT devices 4000 that “trigger” and “cycle” effectively perform discrete phase determination, since the trigger and cycle points are the instants at which the phase changes from exhalation to inhalation and from inhalation to exhalation, respectively. In one implementation of bi-valued phase determination, the phase output Φ is determined to have a discrete value of 0 (thereby “triggering” the RPT device 4000) when the respiratory flow rate Qr has a value that exceeds a positive threshold, and a discrete value of 0.5 revolutions (thereby “cycling” the RPT device 4000) when a respiratory flow rate Qr has a value that is more negative than a negative threshold.

Another implementation of discrete phase determination provides a tri-valued phase output Φ with a value of one of inhalation, mid-inspiratory pause, and exhalation.

In other forms, known as continuous phase determination, the phase output Φ is a continuous value, for example varying from 0 to 1 revolutions, or 0 to 2πradians. RPT devices 4000 that perform continuous phase determination may trigger and cycle when the continuous phase reaches 0 and 0.5 revolutions, respectively. In one implementation of continuous phase determination, a continuous value of phase Φ is determined using a fuzzy logic analysis of the respiratory flow rate Qr. A continuous value of phase determined in this implementation is often referred to as “fuzzy phase”. In one implementation of a fuzzy phase determination algorithm 4321, the following rules are applied to the respiratory flow rate Qr:
1. If the respiratory flow rate is zero and increasing fast then the phase is 0 revolutions.
2. If the respiratory flow rate is large positive and steady then the phase is 0.25 revolutions.
3. If the respiratory flow rate is zero and falling fast, then the phase is 0.5 revolutions.
4. If the respiratory flow rate is large negative and steady then the phase is 0.75 revolutions.
5. If the respiratory flow rate is zero and steady and the 5-second low-pass filtered absolute value of the respiratory flow rate is large then the phase is 0.9 revolutions.
6. If the respiratory flow rate is positive and the phase is expiratory, then the phase is 0 revolutions.
7. If the respiratory flow rate is negative and the phase is inspiratory, then the phase is 0.5 revolutions.
8. If the 5-second low-pass filtered absolute value of the respiratory flow rate is large, the phase is increasing at a steady rate equal to the patient's respiratory rate, low-pass filtered with a time constant of 20 seconds.

The output of each rule may be represented as a vector whose phase is the result of the rule and whose magnitude is the fuzzy extent to which the rule is true. The fuzzy extent to which the respiratory flow rate is “large”, “steady”, etc. is determined with suitable membership functions. The results of the rules, represented as vectors, are then combined by some function such as taking the centroid. In such a combination, the rules may be equally weighted, or differently weighted.

In another implementation of continuous phase determination, the inhalation time Ti and the exhalation time Te are first estimated from the respiratory flow rate Qr. The phase Φ is then determined as the half the proportion of the inhalation time Ti that has elapsed since the previous trigger instant, or 0.5 revolutions plus half the proportion of the exhalation time Te that has elapsed since the previous cycle instant (whichever was more recent).

In some forms of the present technology, suitable for pressure support ventilation therapy (described below), the phase determination algorithm 4321 is configured to trigger even when the respiratory flow rate Qr is insignificant, such as during an apnea. As a result, the RPT device 4000 delivers “backup breaths” in the absence of spontaneous respiratory effort from the patient 1000. For such forms, known as spontaneous/timed (S/T) modes, the phase determination algorithm 4321 may make use of the backup rate Rb provided by the backup rate determination algorithm 4319.

A phase determination algorithm 4321 that uses “fuzzy phase” may implement S/T mode using the backup rate Rb by including a “momentum” rule in the fuzzy phase rules. The effect of the momentum rule is to carry the continuous phase forward from exhalation to inhalation at the backup rate Rb if there are no features of respiratory flow rate Qr that would otherwise carry the continuous phase forward through the other rules. In one implementation, the more it is true that the measure of ventilation Vent (described below) is well below a target value Vtgt for ventilation (also described below), the more highly the momentum rule is weighted in the combination. However, as a result of the rapid increase in pressure support in response to mild to moderate hypoventilation (with respect to the target ventilation), the ventilation may be quite close to the target ventilation. It is desirable that the momentum rule is given a low weighting when the ventilation is close to target, to allow the patient to breathe at rates significantly lower than the respiratory rate at other times (when the patient is not in a central apnea) without being unnecessarily pushed to breathe at a higher rate by the ventilator. However, when the momentum rule is given a low weighting when ventilation is above a value which is below but close to the target ventilation, adequate ventilation may easily be achieved at a relatively high pressure support at a rate well below the backup rate. It would be desirable for the backup breaths to be delivered at a higher rate, because this would enable the target ventilation to be delivered at a lower pressure support. This is desirable for a number of reasons, a key one of which is to diminish mask leak.

To summarise, in a fuzzy phase determination algorithm 4321 that implements S/T mode, there is a dilemma in choosing the weighting for the momentum rule incorporating the backup rate Rb: if it is too high, the patient may feel “pushed along” by the backup rate. If it is too low, the pressure support may be excessive. Hence it is desirable to provide methods of implementing S/T mode which do not rely on the momentum rule described above.

A phase determination algorithm 4321 (either discrete, or continuous without a momentum rule) may implement S/T mode using the backup rate Rb in a manner known as timed backup. Timed backup may be implemented as follows: the phase determination algorithm 4321 attempts to detect the start of inhalation due to spontaneous respiratory effort, for example by monitoring the respiratory flow rate Qr as described above. If the start of inhalation due to spontaneous respiratory effort is not detected within a period of time after the last trigger instant whose duration is equal to the reciprocal of the backup rate Rb (an interval known as the backup timing threshold), the phase determination algorithm 4321 sets the phase output Φ to a value of inhalation (thereby triggering the RPT device 4000). Once the RPT device 4000 is triggered, and a backup breath begins to be delivered, the phase determination algorithm 4321 attempts to detect the start of spontaneous exhalation, for example by monitoring the respiratory flow rate Qr, upon which the phase output Φ is set to a value of exhalation (thereby cycling the RPT device 4000).

If the backup rate Rb is increased over time from the SBR to the STBR, as in a variable backup rate system described above, the backup timing threshold starts out longer and gradually becomes shorter. That is, the RPT device 4000 starts out less vigilant and gradually becomes more vigilant to lack of spontaneous respiratory effort as more backup breaths are delivered. Such an RPT device 4000 is less likely to make a patient feel “pushed along” if they would prefer to breathe at a lower than standard rate, while still delivering backup breaths when they are needed.

If the STBR in a variable backup rate system adapts to the patient's estimated spontaneous respiratory rate Rs, as in an adaptive variable backup rate system described above, the backup breaths will be delivered at a rate that adapts to the patient's own recent spontaneous respiratory efforts.

### 4.3.3.2.2 Waveform Determination

In one form of the present technology, the therapy control module 4330 controls a pressure generator 4140 to provide a treatment pressure Pt that varies as a function of phase Φ of a breathing cycle of a patient according to a waveform template Π(Φ). The waveform template Π(Φ) may be adjusted using any of the aspects described herein, for example, in Sections 1.1-1.2.

In one form of the present technology, a waveform determination algorithm 4322 provides a waveform template Π(Φ) with values in the range [0, 1] on the domain of phase values Φ provided by the phase determination algorithm 4321 to be used by the therapy parameter determination algorithm 4329.

In one form, suitable for either discrete or continuously-valued phase, the waveform template Π(Φ) is a square-wave template, having a value of 1 for values of phase up to and including 0.5 revolutions, and a value of 0 for values of phase above 0.5 revolutions. In one form, suitable for continuously-valued phase, the waveform template Π(Φ) comprises two smoothly curved portions, namely a smoothly curved (e.g., raised cosine) rise from 0 to 1 for values of phase up to 0.5 revolutions, and a smoothly curved (e.g., exponential) decay from 1 to 0 for values of phase above 0.5 revolutions. One example of such a “smooth and comfortable” waveform template is the “shark fin” waveform template, in which the rise is a raised cosine, and the smooth decay is quasi-exponential (so that the limit of Π as Φ approaches one revolution is precisely zero).

In some forms of the present technology, the waveform determination algorithm 4322 selects a waveform template Π(Φ) from a library of waveform templates, dependent on a setting of the RPT device 4000. Each waveform template Π(Φ) in the library may be provided as a lookup table of values Π against phase values Φ. In other forms, the waveform determination algorithm 4322 computes a waveform template Π(Φ) “on the fly” using a predetermined functional form, possibly parametrised by one or more parameters (e.g., time constant of an exponentially curved portion). The parameters of the functional form may be predetermined or dependent on a current state of the patient 1000.

In some forms of the present technology, suitable for discrete bi-valued phase of either inhalation (Φ=0 revolutions) or exhalation (Φ)=0.5 revolutions), the waveform determination algorithm 4322 computes a waveform template Π“on the fly” as a function of both discrete phase Φ and time t measured since the most recent trigger instant (transition from exhalation to inhalation). In one such form, the waveform determination algorithm 4322 computes the waveform template Π(Φ) in two portions (inspiratory and expiratory) as follows:

$$\prod ( Φ , t ) = \{ \begin{matrix}{\prod }_{i} ( t ) , & Φ = 0 \\ {\prod }_{e} ( t - {T}_{i} ) , & Φ = 0. 5\end{matrix}$$

where Πᵢ(t) and Πₑ(t) are inspiratory and expiratory portions of the waveform template Π(Φ, t), and Ti is the inhalation time. In one such form, the inspiratory portion Πᵢ(t) of the waveform template is a smooth rise from 0 to 1 parametrised by a rise time, and the expiratory portion Πₑ(t) of the waveform template is a smooth fall from 1 to 0 parametrised by a fall time.

### 4.3.3.3 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of gas whose mask pressure Pm at the patient interface 3000 is equal to the treatment pressure Pt.

### 4.5 Glossary

For the purposes of the present disclosure, in certain forms of the present technology, one or more of the following definitions may apply. In other forms of the present technology, alternative definitions may apply.

### 4.5.1 General

Air: In certain forms of the present technology, air may be taken to mean atmospheric air, and in other forms of the present technology air may be taken to mean some other combination of breathable gases, e.g., atmospheric air enriched with oxygen.

Respiratory Pressure Therapy (RPT): The delivery of a supply of air to the airways at a treatment pressure that is typically positive with respect to atmosphere.

Continuous Positive Airway Pressure (CPAP) therapy: Respiratory pressure therapy in which the treatment pressure is approximately constant through a breathing cycle of a patient. In some forms, the pressure at the entrance to the airways will be slightly higher during exhalation, and slightly lower during inhalation. In some forms, the pressure will vary between different breathing cycles of the patient, for example, being increased in response to detection of indications of partial upper airway obstruction, and decreased in the absence of indications of partial upper airway obstruction.

Patient: A person, whether or not they are suffering from a respiratory disease.

Automatic Positive Airway Pressure (APAP) therapy: CPAP therapy in which the treatment pressure is automatically adjustable, e.g., from breath to breath, between minimum and maximum limits, depending on the presence or absence of indications of SDB events.

### 4.5.2 Aspects of the Breathing Cycle

Apnea: According to some definitions, an apnea is said to have occurred when respiratory flow rate falls below a predetermined threshold for a duration, e.g., 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort.

Breathing rate, or respiratory rate (Rs): The rate of spontaneous respiration of a patient, usually measured in breaths per minute.

Duty cycle: The ratio of inhalation time, Ti to total breath duration, Ttot.

Effort (breathing): The work done by a spontaneously breathing person attempting to breathe.

Expiratory portion of a breathing cycle: The period from the start of expiratory flow to the start of inspiratory flow.

Flow limitation: The state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Hypopnea: A reduction in flow, but not a cessation of flow. In one form, a hypopnea may be said to have occurred when there is a reduction in flow below a threshold for a duration. In one form in adults, the following either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed.

Positive End-Expiratory Pressure (PEEP): The pressure above atmosphere in the lungs that exists at the end of expiration.

Peak flow rate (Qpeak): The maximum value of flow during the inspiratory portion of the respiratory flow rate waveform.

Respiratory flow/airflow rate, patient flow/airflow rate (Qr): These synonymous terms may be understood to refer to the RPT device's estimate of respiratory airflow rate, as opposed to “true respiratory flow rate” or “true respiratory airflow rate”, which is the actual respiratory flow rate experienced by the patient, usually expressed in litres per minute.

Tidal volume (Vt): The volume of air inhaled or exhaled during normal breathing, when extra effort is not applied.

Inhalation Time (Ti): The duration of the inspiratory portion of the respiratory flow rate waveform.

Exhalation Time (Te): The duration of the expiratory portion of the respiratory flow rate waveform.

(total) Time, or breath duration (Ttot): The total duration between the start of the inspiratory portion of one respiratory flow rate waveform and the start of the inspiratory portion of the following respiratory flow rate waveform.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of the total amount of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 4.5.3 RPT Device Parameters

Flow rate: The instantaneous volume (or mass) of air delivered per unit time. While flow rate and ventilation have the same dimensions of volume or mass per unit time, flow rate is measured over a much shorter period of time. Flow may be nominally positive for the inspiratory portion of a breathing cycle of a patient, and hence negative for the expiratory portion of the breathing cycle of a patient. In some cases, a reference to flow rate will be a reference to a scalar quantity, namely a quantity having magnitude only. In other cases, a reference to flow rate will be a reference to a vector quantity, namely a quantity having both magnitude and direction. Flow rate will be given the symbol Q. ‘Flow rate’ is sometimes shortened to simply ‘flow’. Total flow rate, Qt, is the flow of air leaving the RPT device. Vent flow rate, Qv, is the flow of air leaving a vent to allow washout of exhaled gases. Leak flow rate, Ql, is the flow rate of unintentional leak from a patient interface system. Respiratory flow rate, Qr, is the flow of air that is received into the patient's respiratory system.

Leak: The word leak will be taken to be an unintended flow of air. In one example, leak may occur as the result of an incomplete seal between a mask and a patient's face. In another example leak may occur in a swivel elbow to the ambient.

Pressure: Force per unit area. Pressure may be measured in a range of units, including cmH₂O, g-f/cm², hectopascal. 1 cmH₂O is equal to 1 g-f/cm² and is approximately 0.98 hectopascal. In this specification, unless otherwise stated, pressure is given in units of cmH₂O. The pressure in the patient interface (mask pressure) is given the symbol Pm, while the treatment pressure, which represents a target value to be achieved by the mask pressure Pm at the current instant of time, is given the symbol Pt.

### 4.5.4 Terms for Ventilators

Adaptive Servo-Ventilator (ASV): A servo-ventilator that has a changeable rather than a fixed target ventilation. The changeable target ventilation may be learned from some characteristic of the patient, for example, a respiratory characteristic of the patient.

Backup rate: A parameter of a ventilator that establishes the respiratory rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired mask pressure which the ventilator will attempt to achieve at a given time.

End expiratory pressure (EEP): Desired mask pressure which the ventilator will attempt to achieve at the end of the expiratory portion of the breath. If the pressure waveform template Π(Φ) is zero-valued at the end of expiration, i.e., Π(Φ)=0 when Φ=1, the EEP is equal to the EPAP.

IPAP: desired mask pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP−EPAP). In some contexts pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Servo-assistance: Pressure support minus minimum pressure support.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the inspiratory portion of the breathing cycle by the patient's efforts.

Typical recent ventilation: The typical recent ventilation Vtyp is the value around which recent measures of ventilation over some predetermined timescale tend to cluster, that is, a measure of the central tendency of the measures of ventilation over recent history.

Ventilator: A mechanical device that provides pressure support to a patient to perform some or all of the work of breathing.

### 4.6 Other Remarks

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being preferably used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated by reference to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

Moreover, in interpreting the disclosure, all terms should be interpreted in the broadest reasonable manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular embodiments, it is to be understood that these embodiments are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilized to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative embodiments and that other arrangements may be devised without departing from the spirit and scope of the technology.

Although the present invention has been illustrated by reference to specific embodiments, it will be apparent to those skilled in the art that the invention is not limited to the details of the foregoing illustrative embodiments, and that the present technology may be embodied with various changes and modifications without departing from the scope thereof. The present examples are therefore to be considered in all respects as illustrative and not restrictive, the scope of the technology being indicated by the appended claims rather than by the foregoing description, and all changes which come within the meaning and range of equivalency of the claims are therefore intended to be embraced therein. In other words, it is contemplated to cover any and all modifications, variations or equivalents that fall within the scope of the basic underlying principles and whose essential attributes are claimed in this patent application. It will furthermore be understood by the reader of this patent application that the words “comprising” or “comprise” do not exclude other elements or steps, that the words “a” or “an” do not exclude a plurality, and that a single element, such as a computer system, a processor, or another integrated unit may fulfil the functions of several means recited in the claims. Any reference signs in the claims shall not be construed as limiting the respective claims concerned. The terms “first”, “second”, third”, “a”, “b”, “c”, and the like, when used in the description or in the claims are introduced to distinguish between similar elements or steps and are not necessarily describing a sequential or chronological order. Similarly, the terms “top”, “bottom”, “over”, “under”, and the like are introduced for descriptive purposes and not necessarily to denote relative positions. It is to be understood that the terms so used are interchangeable under appropriate circumstances and embodiments of the technology are capable of operating according to the present technology in other sequences, or in orientations different from the one(s) described or illustrated above.

### 5 Further Examples of the Technology

The following paragraphs further illustrate examples of the present technology described herein.

EXAMPLE 1. A system for providing a respiratory therapy to an airway of a user, comprising:
one or more sensors configured to generate output signals conveying information related to one or more physiological parameters of a user, each of the one or more physiological
parameters are indicative of a degree of user comfort with the respiratory therapy; a pressure generator configured to couple with a patient respiratory interface for delivery of the respiratory therapy to the airway of the user;
a controller coupled to the pressure generator and comprising one or more processors,
wherein the controller is configured to execute a waveform adjustment control loop,
wherein, the controller is configured to, during the waveform adjustment control loop:
   receive output signals from the one or more sensors during delivery of a pressurized flow of breathable gas to the airway of the user in accordance with a predetermined waveform,
   compare each of the one or more physiological parameters to a corresponding baseline value,
   adjust at least one waveform parameter of the predetermined waveform based on the comparison, wherein the adjustment is selected to reduce a difference between a value of at least one of the one or more physiological parameters and the corresponding baseline value for the at least one physiological parameter, and
   operate the pressure generator to generate a pressurized flow of breathable gas in accordance with the predetermined waveform as adjusted.

EXAMPLE 2. The system of EXAMPLE 1, wherein the controller is configured to perform the waveform adjustment control loop iteratively for a predetermined period of time.

EXAMPLE 3. The system of EXAMPLES 1 or 2, wherein the at least one waveform parameter comprises an inspiratory shape of the predetermined waveform.

EXAMPLE 4. The system of EXAMPLE 3, wherein the controller is configured to adjust the predetermined waveform such that the inspiratory shape of the waveform is linear.

EXAMPLE 5. The system of EXAMPLE 3, wherein the controller is configured to adjust the predetermined waveform such that the inspiratory shape is rounded.

EXAMPLE 6. The system of EXAMPLES 1 or 2, wherein the at least one waveform parameter comprises an expiratory shape of the predetermined waveform.

EXAMPLE 7. The system of EXAMPLE 6, wherein the controller is configured to adjust the predetermined waveform such that the expiratory shape of the waveform is linear.

EXAMPLE 8. The system of EXAMPLE 6, wherein the controller is configured to adjust the predetermined waveform such that the expiratory shape of the waveform is rounded.

EXAMPLE 9. The system of EXAMPLE 1, wherein the at least one waveform parameter comprises a rise time of an inspiration phase of the predetermined waveform.

EXAMPLE 10. The system of EXAMPLE 9, wherein the controller is configured to adjust a duration of the rise time.

EXAMPLE 11. The system of EXAMPLE 1, wherein the at least one waveform parameter comprises a fall time of an expiration phase of the predetermined waveform.

EXAMPLE 12. The system of EXAMPLE 11, wherein the controller is configured to adjust a duration of the fall time.

EXAMPLE 13. The system of EXAMPLE 1, wherein the at least one waveform parameter comprises an inspiratory pressure trigger threshold of the predetermined waveform.

EXAMPLE 14. The system of EXAMPLE 1, wherein the at least one waveform parameter comprises an expiratory pressure trigger threshold of the predetermined waveform.

EXAMPLE 15. The system of EXAMPLE 1, wherein the at least one waveform parameter comprises a peak expiratory pressure of the predetermined waveform.

EXAMPLE 16. The system of EXAMPLE 1, wherein the at least one waveform parameter comprises a peak inspiratory pressure of the predetermined waveform.

EXAMPLE 17. The system of any one of EXAMPLES 1 to 16, wherein the one or more physiological parameters comprise one or more of flow rate, pressure, carbon dioxide, tidal volume, breathing rate, breathing effort, heart rate, and movement.

EXAMPLE 18. The system of any one of EXAMPLES 1 to 16, wherein the one or more physiological parameters comprise two or more of flow rate, pressure, carbon dioxide, tidal volume, breathing rate, breathing effort, heart rate, and movement.

EXAMPLE 19. The system of any one of EXAMPLES 1 to 18, wherein the controller is configured to store the predetermined waveform as adjusted in memory.

EXAMPLE 20. A system for providing a respiratory therapy to an airway of a user, comprising:
a pressure generator configured to couple with a patient respiratory interface for delivery of the respiratory therapy to the airway of the user; and
a controller coupled to the pressure generator and comprising one or more processors,
wherein the controller is configured to execute a waveform selection process,
wherein, the controller is configured to, during the waveform selection process:
   provide an indication to a user of delivery of a first waveform in association with operating the pressure generator to generate a pressurized flow of breathable gas in accordance with the first waveform for a first period of time,
   provide an indication to the user of delivery of a second waveform in association with operating the pressure generator to generate a pressurized flow of breathable gas in accordance with the second waveform for a second period of time, and
   with a user interface, prompt for entry of an input selection between the indication of delivery of the first waveform and the indication of delivery of the second waveform.

EXAMPLE 21. The system of EXAMPLE 20, wherein the first waveform is generated in accordance with a first set of one or more waveform parameters and the second waveform is generated in accordance with a second set of one or more waveform parameters, and wherein at least one waveform parameter of the first set is different from at least one waveform parameter of the second set.

EXAMPLE 22. The system of EXAMPLE 21, wherein the different at least one waveform parameter comprises any one of:
an inspiratory shape,
an expiratory shape,
a rise time of an inspiration phase,
a fall time of an expiration phase,
an inspiratory pressure trigger threshold,
an expiratory pressure trigger threshold,
a peak inspiratory pressure,
a peak expiratory pressure,
an inspiratory pressure trigger threshold, and
an expiratory pressure trigger threshold.

EXAMPLE 23. The system of any one of EXAMPLES 20 to 22, wherein the controller is further configured to generate a third waveform based on the entered input selection of the user.

EXAMPLE 24. The system of EXAMPLE 23, wherein the controller is configured to generate the third waveform in accordance with at least one parameter attributable to the selected waveform associated with the entered input selection.

EXAMPLE 25. The system of any one of EXAMPLES 23 to 24, wherein, during the waveform selection process, after generating the third waveform, the controller is further configured to repeat the waveform selection process with (a) the waveform selected by the user between the first waveform and the second waveform and (b) the third waveform.

EXAMPLE 26. The system of EXAMPLE 24, wherein the controller is configured to perform the waveform selection control process iteratively for a predetermined period of time or for a predetermined number of control selection cycles.

EXAMPLE 27. The system of any one of EXAMPLES 20 to 26, wherein the controller is configured to prompt for an indication from the user that the first waveform or the second waveform is at an acceptable level of comfort, and the controller is configured to discontinue the waveform selection control process in response to the indication.

EXAMPLE 28. The system of EXAMPLE 27, wherein, based on the indication from the user, the controller is configured to store the first waveform or the second waveform in memory.

EXAMPLE 29. A system for providing a respiratory therapy to an airway of a user, comprising:
one or more sensors configured to generate output signals conveying information related to one or more physiological parameters of a user, each of the one or more physiological parameters are indicative of at least one aspect of a user's breathing;
a pressure generator configured to couple with a patient respiratory interface for delivery of the respiratory therapy to the airway of the user;
a controller coupled to the pressure generator and comprising one or more processors,
wherein the controller is configured to:
   monitor output signals of the one or more sensors for a period of time,
   approximate the user's breathing pattern based on the monitored output signals,
   generate a personalized pressure waveform based on the approximation of the user's breathing pattern, and
   operate the pressure generator to generate a pressurized flow of breathable gas in accordance with the generated personalized pressure waveform.

EXAMPLE 30. The system of EXAMPLE 29, wherein the one or more sensors compromise one or more of a pressure sensor, a flow sensor, a heart rate sensor, a carbon dioxide sensor, an accelerometer, and one or more EEG sensors.

EXAMPLE 31. The system of any one of EXAMPLES 29 to 30, wherein the one or more physiological parameters comprise one or more of pressure, flow rate, heart rate, carbon dioxide, chest body movement, and lung bioimpedance.

EXAMPLE 32. The system of any one of EXAMPLES 30 to 31, wherein the controller is configured to store one or more parameters of the personalized pressure waveform in memory.

1. A system for providing a respiratory therapy to an airway of a user, comprising:
one or more sensors configured to generate output signals conveying information related to one or more physiological parameters of a user, each of the one or more physiological parameters are indicative of a degree of user comfort with the respiratory therapy;
a pressure generator configured to couple with a patient respiratory interface for delivery of the respiratory therapy to the airway of the user;
a controller coupled to the pressure generator and comprising one or more processors,
wherein the controller is configured to execute a waveform adjustment control loop,
wherein, the controller is configured to, during the waveform adjustment control loop:
receive output signals from the one or more sensors during delivery of a pressurized flow of breathable gas to the airway of the user in accordance with a predetermined waveform,
compare each of the one or more physiological parameters to a corresponding baseline value,
adjust at least one waveform parameter of the predetermined waveform based on the comparison, wherein the adjustment is selected to reduce a difference between a value of at least one of the one or more physiological parameters and the corresponding baseline value for the at least one physiological parameter, and
operate the pressure generator to generate a pressurized flow of breathable gas in accordance with the predetermined waveform as adjusted.

2. The system of claim 1, wherein the controller is configured to perform the waveform adjustment control loop iteratively for a predetermined period of time.

3. The system of claim 1 or 2, wherein the at least one waveform parameter comprises an inspiratory shape of the predetermined waveform.

4. The system of claim 3, wherein the controller is configured to adjust the predetermined waveform such that the inspiratory shape of the waveform is linear.

5. The system of claim 3, wherein the controller is configured to adjust the predetermined waveform such that the inspiratory shape is rounded.

6. The system of claim 1 or 2, wherein the at least one waveform parameter comprises an expiratory shape of the predetermined waveform.

7. The system of claim 6, wherein the controller is configured to adjust the predetermined waveform such that the expiratory shape of the waveform is linear.

8. The system of claim 6, wherein the controller is configured to adjust the predetermined waveform such that the expiratory shape of the waveform is rounded.

9. The system of claim 1, wherein the at least one waveform parameter comprises a rise time of an inspiration phase of the predetermined waveform.

10. The system of claim 9, wherein the controller is configured to adjust a duration of the rise time.

11. The system of claim 1, wherein the at least one waveform parameter comprises a fall time of an expiration phase of the predetermined waveform.

12. The system of claim 11, wherein the controller is configured to adjust a duration of the fall time.

13. The system of claim 1, wherein the at least one waveform parameter comprises an inspiratory pressure trigger threshold of the predetermined waveform.

14. The system of claim 1, wherein the at least one waveform parameter comprises an expiratory pressure trigger threshold of the predetermined waveform.

15. The system of claim 1, wherein the at least one waveform parameter comprises a peak expiratory pressure of the predetermined waveform.

16. The system of claim 1, wherein the at least one waveform parameter comprises a peak inspiratory pressure of the predetermined waveform.

17. The system of any one of claims 1 to 16, wherein the one or more physiological parameters comprise one or more of flow rate, pressure, carbon dioxide, tidal volume, breathing rate, breathing effort, heart rate, and movement.

18. The system of any one of claims 1 to 16, wherein the one or more physiological parameters comprise two or more of flow rate, pressure, carbon dioxide, tidal volume, breathing rate, breathing effort, heart rate, and movement.

19. The system of any one of claims 1 to 18, wherein the controller is configured to store the predetermined waveform as adjusted in memory.

20. A system for providing a respiratory therapy to an airway of a user, comprising:
a pressure generator configured to couple with a patient respiratory interface for delivery of the respiratory therapy to the airway of the user; and
a controller coupled to the pressure generator and comprising one or more processors,
wherein the controller is configured to execute a waveform selection process,
wherein, the controller is configured to, during the waveform selection process:
provide an indication to a user of delivery of a first waveform in association with operating the pressure generator to generate a pressurized flow of breathable gas in accordance with the first waveform for a first period of time,
provide an indication to the user of delivery of a second waveform in association with operating the pressure generator to generate a pressurized flow of breathable gas in accordance with the second waveform for a second period of time, and
with a user interface, prompt for entry of an input selection between the indication of delivery of the first waveform and the indication of delivery of the second waveform.

21. The system of claim 20, wherein the first waveform is generated in accordance with a first set of one or more waveform parameters and the second waveform is generated in accordance with a second set of one or more waveform parameters, and wherein at least one waveform parameter of the first set is different from at least one waveform parameter of the second set.

22. The system of claim 21, wherein the different at least one waveform parameter comprises any one of:
an inspiratory shape,
an expiratory shape,
a rise time of an inspiration phase,
a fall time of an expiration phase,
an inspiratory pressure trigger threshold,
an expiratory pressure trigger threshold,
a peak inspiratory pressure,
a peak expiratory pressure,
an inspiratory pressure trigger threshold, and
an expiratory pressure trigger threshold.

23. The system of any one of claims 20 to 22, wherein the controller is further configured to generate a third waveform based on the entered input selection of the user.

24. The system of claim 23, wherein the controller is configured to generate the third waveform in accordance with at least one parameter attributable to the selected waveform associated with the entered input selection.

25. The system of any one of claims 23 to 24, wherein, during the waveform selection process, after generating the third waveform, the controller is further configured to repeat the waveform selection process with (a) the waveform selected by the user between the first waveform and the second waveform and (b) the third waveform.

26. The system of claim 24, wherein the controller is configured to perform the waveform selection control process iteratively for a predetermined period of time or for a predetermined number of control selection cycles.

27. The system of any one of claims 20 to 26, wherein the controller is configured to prompt for an indication from the user that the first waveform or the second waveform is at an acceptable level of comfort, and the controller is configured to discontinue the waveform selection control process in response to the indication.

28. The system of claim 27, wherein, based on the indication from the user, the controller is configured to store the first waveform or the second waveform in memory.

29. A system for providing a respiratory therapy to an airway of a user, comprising:
one or more sensors configured to generate output signals conveying information related to one or more physiological parameters of a user, each of the one or more physiological parameters are indicative of at least one aspect of a user's breathing;
a pressure generator configured to couple with a patient respiratory interface for delivery of the respiratory therapy to the airway of the user;
a controller coupled to the pressure generator and comprising one or more processors,
wherein the controller is configured to:
monitor output signals of the one or more sensors for a period of time,
approximate the user's breathing pattern based on the monitored output signals,
generate a personalized pressure waveform based on the approximation of the user's breathing pattern, and
operate the pressure generator to generate a pressurized flow of breathable gas in accordance with the generated personalized pressure waveform.

30. The system of claim 29, wherein the one or more sensors compromise one or more of a pressure sensor, a flow sensor, a heart rate sensor, a carbon dioxide sensor, an accelerometer, and one or more EEG sensors.

31. The system of any one of claims 29 to 30, wherein the one or more physiological parameters comprise one or more of pressure, flow rate, heart rate, carbon dioxide, chest body movement, and lung bioimpedance.

32. The system of any one of claims 30 to 31, wherein the controller is configured to store one or more parameters of the personalized pressure waveform in memory.
